(19) United States
(12) Patent Application Publication
(10) Pub. No.: US 2021/0002668 A1
(43) Pub. Date: Jan. 7, 2021

(54) SPHINGOLIPID-METABOLIZING PROTEINS ENHANCE THE EFFICIENCY OF GENE EDITING IN CELLS

(71) Applicant: ICAHN SCHOOL OF MEDICINE AT MOUNT SINAI, New York, NY (US)

(72) Inventors: Efrat ELIYAHU, New York, NY (US); Lior ZANGI, New York, NY (US); Adam VINCEK, New York, NY (US); Yoav HADAS, New York, NY (US)

(21) Appl. No.: 16/978,026

(22) PCT Filed: Mar. 7, 2019
(86) PCT No.: PCT/US2019/021189

§ 371 Date: Sep. 3, 2020

Related U.S. Application Data
(60) Provisional application No. 62/639,718, filed on Mar. 7, 2018.
(60) Provisional application No. 62/692,185, filed on Jun. 29, 2018.

(51) Int. Cl. C12N 15/87; C12N 9/12; C12N 9/80

(52) U.S. Cl. CPC C12N 15/87; C12N 9/1205; C12N 2310/20; C12Y 305/01023; C12Y 207/01091; C12N 9/80

(57) ABSTRACT

The present disclosure pertains to the use of a modified RNA (modRNA) that encodes a sphingolipid-metabolizing protein such as acid ceramidase to achieve expression of the sphingolipid-metabolizing protein in a mammalian cell or group of cells. Expression of the protein from the (modRNA) reduces high levels of ceramide in the cell that lead to cell death or senescence.

### CROSS-REFERENCE TO RELATED APPLICATIONS

This application claims the benefit of U.S. provisional application No. 62/639,718 filed on Mar. 7, 2018, and U.S. provisional application No. 62/692,185 filed Jun. 29, 2018. The contents of each are incorporated by reference in their entirety into the present disclosure.

### SEQUENCE LISTING

The instant application contains a Sequence Listing, created on Mar. 1, 2018; the file, in ASCII format, is designated 3710040AWO_sequencelisting_ST25.txt and is 32.4 kilobytes in size. The file is hereby incorporated by reference in its entirety into the instant application.

### TECHNICAL FIELD

The present disclosure relates generally to gene editing, for example, CRISPR or transcription activator-like effector nuclease (TALEN). Specifically, exposure to or expression of sphingolipid metabolizing proteins by cells undergoing gene editing improves the efficiency of the gene editing.

### BACKGROUND OF THE DISCLOSURE

Gene editing has considerable potential as a therapy for disease with the arrival of genome editing technologies such as zinc-finger nuclease (ZFN), CRISPR/Cas9 and TALEN transfection providing major tools for genome editing.

The process of extracting, genetically modifying and multiplying cells, however, is a huge undertaking and not always scalable. The cells being modified become stressed and vulnerable to apoptotic cell death. The challenges encountered during gene editing include low efficiency (less the 15%), off target deletions and insertions, low embryo survival rates post CRISPR treatment and mosaics formation.

Higher efficiency translates to better success at achieving the desired gene edit result, including, for example in knock-out/knock-in models researchers want and need to explore the function of a gene or a group of genes and move manipulation of those genes to the clinic.

Attempts to improve efficiency by focusing on components of the CRISPR/Cas system enzymes themselves have had limited success. Accordingly, what is needed is a new way to increase the efficiency of the gene editing technology, at the same time avoiding the pitfalls of off-target effects.

### SUMMARY OF THE DISCLOSURE

The present method is designed to improve the efficiency of gene editing by boosting cellular resistance to stress and cell death and initiating a cell survival pathway by reducing the levels of ceramide in the cells. Ceramide levels are diminished by administration to the cells of a sphingolipid-metabolizing protein, such as a ceramidase, or a gene delivery vehicle, such as a modified mRNA (modRNA) that encodes a sphingolipid-metabolizing protein. The improved ability of cells to withstand stress provides an opportunity to enhance the efficiency of gene editing techniques.

In one aspect, therefore, the disclosure relates to a method for improving the efficiency of gene/genome editing of a cell or group of cells by improving the cellular resistance to stress of said cell or group of cells undergoing gene/genome editing. The method comprises contacting a cell or group of cells undergoing gene editing with a sphingolipid-metabolizing protein prior to or concomitantly with gene editing.

In one embodiment, the cell or group of cells is contacted with a modified RNA (modRNA) that encodes a sphingolipid-metabolizing protein selected from the group consisting of (1) ceramidase (2) sphingosine kinase (SPHK), (3) sphingosine-1-phosphate receptor (S1PR) and combinations of modRNAs that encode one of proteins (1), (2) and (3). In one embodiment, cells are primary cells selected from the group consisting of gametes, oocytes, sperm cells, zygotes, stem cells and embryos. The resistance to stress of other mammalian cells undergoing gene editing is encompassed by the disclosure.

In one embodiment, the disclosure relates to a method for improving the efficiency of gene/genome editing comprising culturing a cell or group of cells isolated from a subject in culture medium in the presence of a sphingolipid-metabolizing protein prior to and concurrently with gene/genome editing.

In yet another related aspect, the disclosure relates to a kit comprising one or more modRNAs that encode acid ceramidase (AC), sphingosine kinase (SPHK), and/or sphingosine-1-phosphate receptor (S1PR). In one embodiment, a modRNA that encodes AC has the nucleotide sequence of SEQ ID NO: 1; in another embodiment, a modRNA that encodes AC has the nucleotide sequence of SEQ ID NO: 6; in another embodiment, a modRNA that encodes SPHK1 has the nucleotide sequence of SEQ ID NO: 2; in another embodiment, a modRNA that encodes S1PR has the nucleotide sequence of SEQ ID NO: 3.

In yet another aspect, the disclosure relates to a kit comprising reagents for a gene-editing system and at least one modRNA that encodes a sphingolipid-metabolizing protein. In one embodiment, the sphingolipid-metabolizing protein is selected from the group consisting of acid ceramidase (AC), sphingosine kinase (SPHK), sphingosine-1-phosphate receptor (S1PR) and combinations thereof.

In one embodiment, the gene-editing system is a CRISPR system, for example CRISPR-Cas9.

A modRNA composition useful for this method may include a modRNA encoding a ceramidase, sphingosine kinase (SPHK) modRNA, a sphingosine-1-phosphate receptor (S1PR) modRNA individually or in different combinations thereof. Ceramidase is the only enzyme that can regulate ceramide hydrolysis to prevent cell death and SPHK is the only enzyme that can synthesize Sphingosine 1 Phosphate (S1P) from Sphingosine (the ceramide hydrolysis product) to initiate cell survival. S1PR, a G protein-coupled receptor binds the lipid-signaling molecule S1P to induce cell proliferation, survival, and transcriptional activation.

modRNA is a synthetic mRNA with optimized 5′UTR and 3′UTR sequences, anti-reverse cup analog (ARCA) and one or more naturally modified nucleotides. The optimized UTRs sequences enhance the translation efficiency. ARCA increase the stability of the RNA and enhance the translation efficiency and the naturally modified nucleotides increase the stability of the RNA reduce the innate immune response of cells (in vitro and in vivo) and enhance the translation efficiency of the mRNA. This combination generates a superior mRNA that mediates a higher and longer expression of proteins with a minimal immune response. Modified mRNA has been shown to be a safe, local, transient, and high expression gene delivery method

Not wishing to be bound by theory, the present invention provides a method for improving the efficiency of gene editing by inhibiting apoptotic death of the cells being treated and initiating a survival pathway in those cells, thereby prolonging the life span of cells cultured in vitro by administration of a sphingolipid-metabolizing protein such as ceramidase or modified mRNAs (modRNA) and other vectors that encode sphingolipid-metabolizing proteins.

In one embodiment, the disclosure relates to a method to improve gene editing efficiency, the method comprising contacting said cell or group of cells with a modified RNA (modRNA) selected from the group consisting of (1) modRNA that encodes ceramidase (2) modRNA that encodes sphingosine kinase (SPHK), (3) modified RNA (modRNA) that encodes sphingosine-1-phosphate receptor (S1PR) and combinations of (1), (2) and (3). Cells are mammalian cells and may be selected from the group consisting of primary cells (for example hematopoietic cells), gametes, oocytes, sperm cells, zygotes, embryos and stem cells.

In a related aspect, the disclosure relates to a method to improve efficiency of gene editing of oocytes and/or embryos in vitro, comprising contacting said oocytes or embryos with (1) modRNA that encodes ceramidase, (2) modRNA that encodes sphingosine kinase (SPHK), (3) modified RNA (modRNA) that encodes sphingosine-1-phosphate receptor (S1PR) or any combination of (1), (2), and (3).

In yet another related aspect, the disclosure relates to a composition comprising one or more of modRNA that encodes ceramidase, modRNA that encodes sphingosine kinase (SPHK), and modRNA that encodes sphingosine-1-phosphate receptor (S1PR).

In one embodiment the modRNA encodes acid ceramidase and has the oligonucleotide sequence of SEQ ID NO: 1. In another embodiment, the modRNA encoding AC has the oligonucleotide sequence of SEQ ID NO: 6. In another embodiment, the cells are contacted with a modRNA that encodes sphingosine kinase (SPHK) having the oligonucleotide sequence of SEQ ID NO: 2. In another embodiment, the sphingolipid metabolizing molecule is S1PR and the oligonucleotide encoding it has the sequence SEQ ID NO: 3.

In one aspect, the present disclosure relates to a method to improve quality/survival of cells comprising contacting said cells with a (1) modRNA that encodes ceramidase, (2) modRNA that encodes sphingosine kinase (SPHK), (3) modified RNA (modRNA) that encodes sphingosine-1-phosphate receptor (S1PR) or any combination of (1), (2), and (3).

In some embodiments, the cells are mammalian cells. In some embodiments, the cells are selected from the group consisting of primary cell lines, stem cells, in vitro or in vivo or oocytes and/or embryos in culture.

Compositions comprising any combination of modRNAs that encode (1) ceramidase, (2) sphingosine kinase (SPHK), (3) sphingosine-1-phosphate receptor (S1PR) are encompassed by the present disclosure.

### DETAILED DESCRIPTION OF THE DISCLOSURE

All patents, published applications and other references cited herein are hereby incorporated by reference into the present application.

In the description that follows, certain conventions will be followed as regards the usage of terminology. In general, terms used herein are intended to be interpreted consistently with the meaning of those terms as they are known to those of skill in the art. Some definitions are provided purely for the convenience of the reader.

The term “cell or group of cells” is intended to encompass single cells as well as a plurality of cells either in suspension or in monolayers. Whole tissues also constitute a group of cells.

The term “cell quality” or “quality of a cell” refers to the standard of cell viability, and cellular function as measured against a normal healthy cell with normal cell function and expected life span, the quality of cells that are programmed for survival but not for cell death.

The term “modRNA” refers to a synthetic modified RNA that can be used for expression of a gene of interest. Chemical modifications made in the modRNA, for example, substitution of uridine with pseudouridine, stabilizes the molecule and enhances transcription. Additionally, unlike delivery of protein agents directly to a cell, which can activate the immune system, the delivery of modRNA can be achieved without immune impact. The use of modRNA for in vivo and in vitro expression is described in more detail in for example, WO 2012/138453.

The term “CRISPR/CAS”, “clustered regularly interspaced short palindromic repeats system,” or “CRISPR” refers to DNA loci-containing short repetitions of base sequences. Each repetition is followed by short segments of spacer DNA from previous exposures to a virus. Bacteria and archaea have evolved the use of short RNA sequences to direct degradation of foreign nucleic acids as an adaptive immune defense. Methods for gene editing by CRISPR/Cas are well known to those of skill in the art. The improvement to gene editing afforded by the presently disclosed method is improving the overall condition of the cells to be edited, thereby providing a larger pool of cells that are potentially successfully editable. The use of modRNA to achieve expression of sphingolipid-metabolizing proteins improves survival and DNA repair of cells undergoing gene editing, which lowers the chances for off-target deletions and insertions.

The term “efficiency of gene editing” or “improvement in gene editing” refers to the improved ability to achieve successful gene editing (positive editing), ostensibly by improving cell survival and DNA repair, while avoiding unintended effects such as off-target deletions or insertions. Efficiency rates, therefore, pertain to the number of cells successfully edited.

The present disclosure relates to a method for improving the efficiency of gene editing. In one embodiment, the gene editing platform used is CRISPR/Cas9. The method is built on the premise that overall gene editing efficiency can be improved by making the cells to be edited more robust and more resistant to stress that they experience as a result of gene editing.

In one embodiment, the method relies on the use of a sphingolipid-metabolizing protein, such as ceramidase, to reduce ceramide levels in cells that are undergoing gene editing.

In one embodiment, the method relies on the use of a gene delivery modality such as modRNA for expressing sphingolipid-metabolizing proteins to reduce ceramide levels in cells that are undergoing gene editing, thereby reducing ceramide levels as a result of cellular stress and induction of cell death in those cells. The method results in a higher number of successfully gene-edited cells compared to cells that do not express a sphingolipid-metabolizing protein.

Apoptosis, programmed cell death, is an important physiological process controlling the life span of all cells in vitro and in vivo. The ability to control apoptosis therefore, may be important therapeutically.

Use of Ceramidase Protein or modRNA Encoding Ceramidase to Improve Gene-Editing Efficiency

Ceramide, SPH and S1P are bioactive lipids that mediate cell proliferation, differentiation, apoptosis, adhesion and migration. High levels of cellular ceramides can trigger programmed cell death while ceramide metabolites such as ceramide 1 phosphate and sphingosine 1 phosphate are associated with cell survival and proliferation.

For example, myocardial infarction (MI) is an acute life threatening medical condition. It is caused by blockage of a coronary artery which leads to ischemia and later to necrosis of the affected heart area. Several strategies have been proposed as potential treatment to post MI including the inhibition of controlled cell death.

In MI, the level of lipids in the patient's blood during acute MI can serve to predict the risk for complication. In particular, high levels of ceramides has been associated with a higher probability of recurring events and mortality.

Similarly, a major challenge of assisted reproduction technologies (ARTs) is to mimic the natural environment required to sustain oocyte and embryo survival. There are several studies that support association of ceramide with cellular and organismal aging, which among other things, impacts reproduction.

Under normal physiological conditions 85-90% of oocytes succumb to apoptosis at some point during fetal or postnatal life. Ovulated oocytes undergo molecular changes characteristic of apoptosis unless successful fertilization occurs. While multiple factors, including ceramide, have been characterized as pro-apoptotic elements involved in this process, little is known about factors that sustain oocyte/embryo survival.

Thus, an approach for reducing ceramide levels was developed based on a strategy of inducing transient expression and avoiding integration of the vector into the genome of the recipient cell.

The use of unmodified exogenous RNA as a gene delivery method is ineffective due to its instability outside the cell and the strong innate immune response it elicits when transfected into cells. The discovery by Kariko et al. (Incorporation of Pseudouridine Into mRNA Yields Superior Nonimunogenic Vector With Increased Translational Capacity and Biological Stability. Mol Ther. 2008; 16(11): 1833-1840, incorporated herein by reference) that the substitution of uridine and cytidine with pseudouridine and 5-methylcytidine, respectively drastically reduced the immune response elicited from exogenous RNA and set the stage for a new kind of gene delivery, in which transient expression of therapeutic proteins is achieved.

Additionally, depending on the cell type and indication for which ceramide reduction is needed, delivery of a nucleic acid that encodes a sphingolipid-metabolizing enzyme to cells can be achieved with a modRNA that encodes a sphingolipid-metabolizing enzyme.

Therefore, the present disclosure provides a method for improving the efficiency of gene/genome editing the cells to be edited by contacting said cells with a sphingolipid-metabolizing protein or a modRNA that encodes the sphingolipid-metabolizing protein to inhibit cell death and initiate survival, thereby promoting cell quality and cell survival. The choice of delivery method will depend on cell type and desired duration of expression.

### ModRNA Delivery

Modified mRNA (modRNA) is a relatively new gene delivery system, which can be used in vitro or in vivo to achieve transient expression of therapeutic proteins in a heterogeneous population of cells. Incorporation of specific modified nucleosides enables modRNA to be translated efficiently without triggering antiviral and innate immune responses. In the present disclosure, modRNA is shown to be effective at delivering short-term robust gene expression of a “survival gene”. A stepwise protocol for the synthesis of modRNA for delivery of therapeutic proteins is disclosed in, for example, Kondrat et al. Synthesis of Modified mRNA for Myocardial Delivery. Cardiac Gene Therapy, pp. 127-138 2016, the contents of which are hereby incorporated by reference into the present disclosure.

### Shingolipid-Metabolizing Proteins

A composition useful for the method of the present disclosure may include either individually or in different combinations modRNAs encoding the following sphingolipid-metabolizing proteins: ceramidase, sphingosine kinase (SPHK), and sphingosine-1-phosphate receptor (S1PR). In one embodiment, the sphingolipid-metabolizing protein is a ceramidase.

Ceramidase is an enzyme that cleaves fatty acids from ceramide, producing sphingosine (SPH), which in turn is phosphorylated by a sphingosine kinase to form sphingosine-1-phosphate (S1P). Ceramidase is the only enzyme that can regulate ceramide hydrolysis to prevent cell death and SHPK is the only enzyme that can synthesize sphingosine 1 phosphate (S1P) from sphingosine (the ceramide hydrolysis product) to initiate cell survival. S1PR, a G protein-coupled receptor binds the lipid-signaling molecule S1P to induce cell proliferation, survival, and transcriptional activation.

Presently, 7 human ceramidases encoded by 7 distinct genes have been cloned:
acid ceramidase (ASAH1)—associated with cell survival;
neutral ceramidase (ASAH2, ASAH2B, ASAH2C)—protective against inflammatory cytokines;
alkaline ceramidase 1 (ACER1)—mediating cell differentiation by controlling the generation of SPH and S1P;
alkaline ceramidase 2 (ACER2)—important for cell proliferation and survival; and
alkaline ceramidase 3 (ACER3).

Table 1 contains nucleotide sequences that encode sphingolipid metabolizing proteins of the present method.

TABLE 1

| Gene | Open Reading Frame |
|---|---|
| ASAH1 | ATGCCGGGCCGGAGTTGCGTCGCCTTAGTCCTCCTGGCTGCCGCCGTCAGCTGTGCCGTCGCGCA |
| transcript | GCACGCGCCGCCGTGGACAGAGGACTGCAGAAAATCAACCTATCCTCCTTCAGGACCAACGTAC |
| variant 1 | AGAGGTGCAGTTCCATGGTACACCATAAATCTTGACTTACCACCCTACAAAAGATGGCATGAATT |
| (ACv1) | GATGCTTGACAAGGCACCAGTGCTAAAGGTTATAGTGAATTCTCTGAAGAATATGATAAATACAT |
| | TCGTGCCAAGTGGAAAAATTATGCAGGTGGTGGATGAAAAATTGCCTGGCCTACTTGGCAACTTT |
| | CCTGGCCCTTTTGAAGAGGAAATGAAGGGTATTGCCGCTGTTACTGATATACCTTTAGGAGAGAT |
| | TATTTCATTCAATATTTTTTATGAATTATTTACCATTTGTACTTCAATAGTAGCAGAAGACAAAAAA |
| | GGTCATCTAATACATGGGAGAAACATGGATTTTGGAGTATTTCTTGGGTGGAACATAAATAATGA |
| | TACCTGGGTCATAACTGAGCAACTAAAACCTTTAACAGTGAATTTGGATTTCCAAAGAAACAACA |
| | AAACTGTCTTCAAGGCTTCAAGCTTTGCTGGCTATGTGGGCATGTTAACAGGATTCAAACCAGGA |
| | CTGTTCAGTCTTACACTGAATGAACGTTTCAGTATAAATGGTGGTTATCTGGGTATTCTAGAATGG |
| | ATTCTGGGAAAGAAAGATGTCATGTGGATAGGGTTCCTCACTAGAACAGTTCTGGAAAATAGCA |
| | CAAGTTATGAAGAAGCCAAGAATTTATTGACCAAGACCAAGATATTGGCCCCAGCCTACTTTATC |
| | CTGGGAGGCAACCAGTCTGGGGAAGGTTGTGTGATTACACGAGACAGAAAGGAATCATTGGAT |
| | GTATATGAACTCGATGCTAAGCAGGGTAGATGGTATGTGGTACAAACAAATTATGACCGTTGGA |
| | AACATCCCTTCTTCCTTGATGATCGCAGAACGCCTGCAAAGATGTGTCTGAACCGCACCAGCCAA |
| | GAGAATATCTCATTTGAAACCATGTATGATGTCCTGTCAACAAAACCTGTCCTCAACAAGCTGACC |
| | GTATACACAACCTTGATAGATGTTACCAAAGGTCAATTCGAAACTTACCTGCGGGACTGCCCTGA |
| | CCCTTGTATAGGTTGGTGA (SEQ ID NO: 1) |
| Sphk1 | ATGGATCCAGTGGTCGGTTGCGGACGTGGCCTCTTTGGTTTTGTTTTCTCAGCGGGCGGCCCCCG |
| | GGGCGTGCTCCCGCGGCCCTGCCGCGTGCTGGTGCTGCTGAACCCGCGCGGCGGCAAGGGCAA |
| | GGCCTTGCAGCTCTTCCGGAGTCACGTGCAGCCCCTTTTGGCTGAGGCTGAAATCTCCTTCACGCT |
| | GATGCTCACTGAGCGGCGGAACCACGCGCGGGAGCTGGTGCGGTCGGAGGAGCTGGGCCGCTG |
| | GGACGCTCTGGTGGTCATGTCTGGAGACGGGCTGATGCACGAGGTGGTGAACGGGCTCATGGA |
| | GCGGCCTGACTGGGAGACCGCCATCCAGAAGCCCCTGTGTAGCCTCCCAGCAGGCTCTGGCAAC |
| | GCGCTGGCAGCTTCCTTGAACCATTATGCTGGCTATGAGCAGGTCACCAATGAAGACCTCCTGAC |
| | CAACTGCACGCTATTGCTGTGCCGCCGGCTGCTGTCACCCATGAACCTGCTGTCTCTGCACACGGC |
| | TTCGGGGCTGCGCCTCTTCTCTGTGCTCAGCCTGGCCTGGGGCTTCATTGCTGATGTGGACCTAG |
| | AGAGTGAGAAGTATCGGCGTCTGGGGGAGATGCGCTTCACTCTGGGCACCTTCCTGCGTCTGGC |
| | AGCCCTGCGCACCTACCGCGGCCGACTGGCCTACCTCCCTGTAGGAAGAGTGGGTTCCAAGACAC |
| | CTGCCTCCCCCGTTGTGGTCCAGCAGGGCCCGGTAGATGCACACCTTGTGCCACTGGAGGAGCCA |
| | GTGCCCTCTCACTGGACAGTGGTGCCCGACGAGGACTTTGTGCTAGTCCTGGCACTGCTGCACTC |
| | GCACCTGGGCAGTGAGATGTTTGCTGCACCCATGGGCCGCTGTGCAGCTGGCGTCATGCATCTGT |
| | TCTACGTGCGGGCGGGAGTGTCTCGTGCCATGCTGCTGCGCCTCTTCCTGGCCATGGAGAAGGG |
| | CAGGCATATGGAGTATGAATGCCCCTACTTGGTATATGTGCCCGTGGTCGCCTTCCGCTTGGAGC |
| | CCAAGGATGGGAAAGGTGTGTTTGCAGTGGATGGGGAATTGATGGTTAGCGAGGCCGTGCAGG |
| | GCCAGGTGCACCCAAACTACTTCTGGATGGTCAGCGGTTGCGTGGAGCCCCCGCCCAGCTGGAA |
| | GCCCCAGCAGATGCCACCGCCAGAAGAGCCCTTATGA (SEQ ID NO: 2) |
| S1PR2 | ATGGGCAGCTTGTACTCGGAGTACCTGAACCCCAACAAGGTCCAGGAACACTATAATTATACCAA |
| | GGAGACGCTGGAAACGCAGGAGACGACCTCCCGCCAGGTGGCCTCGGCCTTCATCGTCATCCTCT |
| | GTTGCGCCATTGTGGTGGAAAACCTTCTGGTGCTCATTGCGGTGGCCCGAAACAGCAAGTTCCAC |
| | TCGGCAATGTACCTGTTTCTGGGCAACCTGGCCGCCTCCGATCTACTGGCAGGCGTGGCCTTCGT |
| | AGCCAATACCTTGCTCTCTGGCTCTGTCACGCTGAGGCTGACGCCTGTGCAGTGGTTTGCCCGGG |
| | AGGGCTCTGCCTTCATCACGCTCTCGGCCTCTGTCTTCAGCCTCCTGGCCATCGCCATTGAGCGCC |
| | ACGTGGCCATTGCCAAGGTCAAGCTGTATGGCAGCGACAAGAGCTGCCGCATGCTTCTGCTCATC |
| | GGGGCCTCGTGGCTCATCTCGCTGGTCCTCGGTGGCCTGCCCATCCTTGGCTGGAACTGCCTGGG |
| | CCACCTCGAGGCCTGCTCCACTGTCCTGCCTCTCTACGCCAAGCATTATGTGCTGTGCGTGGTGAC |
| | CATCTTCTCCATCATCCTGTTGGCCATCGTGGCCCTGTACGTGCGCATCTACTGCGTGGTCCGCTC |
| | AAGCCACGCTGACATGGCCGCCCCGCAGACGCTAGCCCTGCTCAAGACGGTCACCATCGTGCTAG |
| | GCGTCTTTATCGTCTGCTGGCTGCCCGCCTTCAGCATCCTCCTTCTGGACTATGCCTGTCCCGTCCA |
| | CTCCTGCCCGATCCTCTACAAAGCCCACTACTTTTTCGCCGTCTCCACCCTGAATTCCCTGCTCAAC |
| | CCCGTCATCTACACGTGGCGCAGCCGGGACCTGCGGCGGGAGGTGCTTCGGCCGCTGCAGTGCT |
| | GGAGGCCGGGGGTGGGGGTGCAAGGACGGAGGCGGGGCGGGACCCCGGGCCACCACCTCCTG |
| | CCACTCCGCAGCTCCAGCTCCCTGGAGAGGGGCATGCACATGCCCACGTCACCCACGTTTCTGGA |
| | GGGCAACACGGTGGTCATG (SEQ ID NO: 3) |
| Firefly | ATGGCCGATGCTAAGAACATTAAGAAGGGCCCTGCTCCCTTCTACCCTCTGGAGGATGGCACCGC |
| luciferase | TGGCGAGCAGCTGCACAAGGCCATGAAGAGGTATGCCCTGGTGCCTGGCACCATTGCCTTCACC |
| | GATGCCCACATTGAGGTGGACATCACCTATGCCGAGTACTTCGAGATGTCTGTGCGCCTGGCCGA |
| | GGCCATGAAGAGGTACGGCCTGAACACCAACCACCGCATCGTGGTGTGCTCTGAGAACTCTCTGC |
| | AGTTCTTCATGCCAGTGCTGGGCGCCCTGTTCATCGGAGTGGCCGTGGCCCCTGCTAACGACATT |
| | TACAACGAGCGCGAGCTGCTGAACAGCATGGGCATTTCTCAGCCTACCGTGGTGTTCGTGTCTAA |
| | GAAGGGCCTGCAGAAGATCCTGAACGTGCAGAAGAAGCTGCCTATCATCCAGAAGATCATCATC |
| | ATGGACTCTAAGACCGACTACCAGGGCTTCCAGAGCATGTACACATTCGTGACATCTCATCTGCCT |
| | CCTGGCTTCAACGAGTACGACTTCGTGCCAGAGTCTTTCGACAGGGACAAAACCATTGCCCTGAT |
| | CATGAACAGCTCTGGGTCTACCGGCCTGCCTAAGGGCGTGGCCCTGCCTCATCGCACCGCCTGTG |
| | TGCGCTTCTCTCACGCCCGCGACCCTATTTTCGGCAACCAGATCATCCCCGACACCGCTATTCTGA |
| | GCGTGGTGCCATTCCACCACGGCTTCGGCATGTTCACCACCCTGGGCTACCTGATTTGCGGCTTTC |
| | GGGTGGTGCTGATGTACCGCTTCGAGGAGGAGCTGTTCCTGCGCAGCCTGCAAGACTACAAAAT |
| | TCAGTCTGCCCTGCTGGTGCCAACCCTGTTCAGCTTCTTCGCTAAGAGCACCCTGATCGACAAGTA |
| | CGACCTGTCTAACCTGCACGAGATTGCCTCTGGCGGCGCCCCACTGTCTAAGGAGGTGGGCGAA |
| | GCCGTGGCCAAGCGCTTTCATCTGCCAGGCATCCGCCAGGGCTACGGCCTGACCGAGACAACCA |
| | GCGCCATTCTGATTACCCCAGAGGGCGACGACAAGCCTGGCGCCGTGGGCAAGGTGGTGCCATT |
| | CTTCGAGGCCAAGGTGGTGGACCTGGACACCGGCAAGACCCTGGGAGTGAACCAGCGCGGCGA |
| | GCTGTGTGTGCGCGGCCCTATGATTATGTCCGGCTACGTGAATAACCCTGAGGCCACAAACGCCC |
| | TGATCGACAAGGACGGCTGGCTGCACTCTGGCGACATTGCCTACTGGGACGAGGACGAGCACTT |
| | CTTCATCGTGGACCGCCTGAAGTCTCTGATCAAGTACAAGGGCTACCAGGTGGCCCCAGCCGAGC |
| | TGGAGTCTATCCTGCTGCAGCACCCTAACATTTTCGACGCCGGAGTGGCCGGCCTGCCCGACGAC |
| | GATGCCGGCGAGCTGCCTGCCGCCGTCGTCGTGCTGGAACACGGCAAGACCATGACCGAGAAG |
| | GAGATCGTGGACTATGTGGCCAGCCAGGTGACAACCGCCAAGAAGCTGCGCGGCGGAGTGGTG |
| | TTCGTGGACGAGGTGCCCAAGGGCCTGACCGGCAAGCTGGACGCCCGCAAGATCCGCGAGATCC |
| | TGATCAAGGCTAAGAAAGGCGGCAAGATCGCCGTGTAA (SEQ ID NO: 4) |
| nGFP | ATGGTGAGCAAGGGCGAGGAGCTGTTCACCGGGGTGGTGCCCATCCTGGTCGAGCTGGACGGC |
| | GACGTAAACGGCCACAAGTTCAGCGTGTCCGGCGAGGGCGAGGGCGATGCCACCTACGGCAAG |
| | CTGACCCTGAAGTTCATCTGCACCACCGGCAAGCTGCCCGTGCCCTGGCCCACCCTCGTGACCACC |
| | CTGACCTACGGCGTGCAGTGCTTCAGCCGCTACCCCGACCACATGAAGCAGCACGACTTCTTCAA |
| | GTCCGCCATGCCCGAAGGCTACGTCCAGGAGCGCACCATCTTCTTCAAGGACGACGGCAACTACA |
| | AGACCCGCGCCGAGGTGAAGTTCGAGGGCGACACCCTGGTGAACCGCATCGAGCTGAAGGGCA |
| | TCGACTTCAAGGAGGACGGCAACATCCTGGGGCACAAGCTGGAGTACAACTACAACAGCCACAA |
| | CGTCTATATCATGGCCGACAAGCAGAAGAACGGCATCAAGGTGAACTTCAAGATCCGCCACAAC |
| | ATCGAGGACGGCAGCGTGCAGCTCGCCGACCACTACCAGCAGAACACCCCCATCGGCGACGGCC |
| | CCGTGCTGCTGCCCGACAACCACTACCTGAGCACCCAGTCCGCCCTGAGCAAAGACCCCAACGAG |
| | AAGCGCGATCACATGGTCCTGCTGGAGTTCGTGACCGCCGCCGGGATCACTCTCGGCATGGACG |
| | AGCTGTACAAGGGAGATCCAAAAAAGAAGAGAAAGGTAGGCGATCCAAAAAAGAAGAGAAAG |
| | GTAGGTGATCCAAAAAAGAAGAGAAAGGTATAA (SEQ ID NO: 5) |
| ASAH2 | ATGAACTGCTGCATCGGGCTGGGAGAGAAAGCTCGCGGGTCCCACCGGGCCTCCTACCCAAGTC |
| transcript | TCAGCGCGCTTTTCACCGAGGCCTCAATTCTGGGATTTGGCAGCTTTGCTGTGAAAGCCCAATGG |
| variant 2 | ACAGAGGACTGCAGAAAATCAACCTATCCTCCTTCAGGACCAACGTACAGAGGTGCAGTTCCATG |
| (ACv2) | GTACACCATAAATCTTGACTTACCACCCTACAAAAGATGGCATGAATTGATGCTTGACAAGG |
| | CAGTGCTAAAGGTTATAGTGAATTCTCTGAAGAATATGATAAATACATTCGTGCCAAGTGGAAAACAC |
| | ATTATGCAGGTGGTGGATGAAAAATTGCCTGGCCTACTTGGCAACTTTCCTGGCCCTTTTGAAGA |
| | GGAAATGAAGGGTATTGCCGCTGTTACTGATATACCTTTAGGAGAGATTATTTCATTCAATATTTT |
| | TTATGAATTATTTACCATTTGTACTTCAATAGTAGCAGAAGACAAAAAAGGTCATCTAATACATGG |
| | GAGAAACATGGATTTTGGAGTATTTCTTGGGTGGAACATAAATAATGATACCTGGGTCATAACTG |
| | AGCAACTAAAACCTTTAACAGTGAATTTGGATTTCCAAAGAAACAACAAAACTGTCTTCAAGGCTT |
| | CAAGCTTTGCTGGCTATGTGGGCATGTTAACAGGATTCAAACCAGGACTGTTCAGTCTTACACTG |
| | AATGAACGTTTCAGTATAAATGGTGGTTATCTGGGTATTCTAGAATGGATTCTGGGAAAGAAAGA |
| | TGTCATGTGGATAGGGTTCCTCACTAGAACAGTTCTGGAAAATAGCACAAGTTATGAAGAAGCCA |
| | AGAATTTATTGACCAAGACCAAGATATTGGCCCCAGCCTACTTTATCCTGGGAGGCAACCAGTCT |
| | GGGGAAGGTTGTGTGATTACACGAGACAGAAAGGAATCATTGGATGTATATGAACTCGATGCTA |
| | AGCAGGGTAGATGGTATGTGGTACAAACAAATTATGACCGTTGGAAACATCCCTTCTTCCTTGAT |
| | GATCGCAGAACGCCTGCAAAGATGTGTCTGAACCGCACCAGCCAAGAGAATATCTCATTTGAAAC |
| | CATGTATGATGTCCTGTCAACAAAACCTGTCCTCAACAAGCTGACCGTATACACAACCTTGATAGA |
| | TGTTACCAAAGGTCAATTCGAAACTTACCTGCGGGACTGCCCTGACCCTTGTATAGGTTGGTGA |
| | (SEQ ID NO: 6) |
| ASAH 1 | ATGAACTGCTGCATCGGGCTGGGAGAGAAAGCTCGCGGGTCCCACCGGGCCTCCTACCCAAGTC |
| transcript | TCAGCGCGCTTTTCACCGAGGCCTCAATTCTGGGATTTGGCAGCTTTGCTGTGAAAGCCCAATGG |
| variant 3 | ACAGAGGACTGCAGAAAATCAACCTATCCTCCTTCAGGACCAACTGTCTTCCCTGCTGTTATAAGG |
| | TACAGAGGTGCAGTTCCATGGTACACCATAAATCTTGACTTACCACCCTACAAAAGATGGCATGA |
| | ATTGATGCTTGACAAGGCACCAGTGCCTGGCCTACTTGGCAACTTTCCTGGCCCTTTTGAAGAGG |
| | AAATGAAGGGTATTGCCGCTGTTACTGATATACCTTTAGGAGAGATTATTTCATTCAATATTTTTT |
| | ATGAATTATTTACCATTTGTACTTCAATAGTAGCAGAAGACAAAAAAGGTCATCTAATACATGGG |
| | AGAAACATGGATTTTGGAGTATTTCTTGGGTGGAACATAAATAATGATACCTGGGTCATAACTGA |
| | GCAACTAAAACCTTTAACAGTGAATTTGGATTTCCAAAGAAACAACAAAACTGTCTTCAAGGCTTC |
| | AAGCTTTGCTGGCTATGTGGGCATGTTAACAGGATTCAAACCAGGACTGTTCAGTCTTACACTGA |
| | ATGAACGTTTCAGTATAAATGGTGGTTATCTGGGTATTCTAGAATGGATTCTGGGAAAGAAAGAT |
| | GTCATGTGGATAGGGTTCCTCACTAGAACAGTTCTGGAAAATAGCACAAGTTATGAAGAAGCCA |
| | AGAATTTATTGACCAAGACCAAGATATTGGCCCCAGCCTACTTTATCCTGGGAGGCAACCAGTCT |
| | GGGGAAGGTTGTGTGATTACACGAGACAGAAAGGAATCATTGGATGTATATGAACTCGATGCTA |
| | AGCAGGGTAGATGGTATGTGGTACAAACAAATTATGACCGTTGGAAACATCCCTTCTTCCTTGAT |
| | GATCGCAGAACGCCTGCAAAGATGTGTCTGAACCGCACCAGCCAAGAGAATATCTCATTTGAAAC |
| | CATGTATGATGTCCTGTCAACAAAACCTGTCCTCAACAAGCTGACCGTATACACAACCTTGATAGA |
| | TGTTACCAAAGGTCAATTCGAAACTTACCTGCGGGACTGCCCTGACCCTTGTATAGGTTGGTGA |
| | (SEQ ID NO: 7) |
| ASAH2 | ATGGGCCAAACGCACCTTCTCTAACTTGGAGACATTCCTGATTTTCCTCCTTGTAATGATGAGTGCC |
| transcript | ATCACAGTGGCCCTTCTCAGCCTCTTGTTTATCACCAGTGGGACCATTGAAAACCACAAAGATTTA |
| variant 1 | GGAGGCCATTTTTTTTCAACCACCCAAAGCCCTCCAGCCACCCAGGGCTCCACAGCTGCCCAACGC |
| | TCCACAGCCACCCAGCATTCCACAGCCACCCAGAGCTCCACAGCCACTCAAACTTCTCCAGTGCCT |
| | TTAACCCCAGAGTCTCCTCTATTTCAGAACTTCAGTGGCTACCATATTGGTGTTGGACGAGCTGAC |
| | TGCACAGGACAAGTAGCAGATATCAATTTGATGGGCTATGGCAAATCCGGCCAGAATGCACAGG |
| | GCATCCTCACCAGGCTATACAGTCGTGCCTTCATCATGGCAGAACCTGATGGGTCCAATCGAACA |
| | GTGTTTGTCAGCATCGACATAGGCATGGTATCACAAAGGCTCAGGCTGGAGGTCCTGAACAGAC |
| | TGCAGAGTAAATATGGCTCCCTGTACAGAAGAGATAATGTCATCCTGAGTGGCACTCACACTCAT |
| | TCAGGTCCTGCAGGATATTTCCAGTATACCGTGTTTGTAATTGCCAGTGAAGGATTTAGCAATCAA |
| | ACTTTTCAGCACATGGTCACTGGTATCTTGAAGAGCATTGACATAGCACACACAAATATGAAACC |
| | AGGCAAAATCTTCATCAATAAAGGAAATGTGGATGGTGTGCAGATCAACAGAAGTCCGTATTCTT |
| | ACCTTCAAAATCCGCAGTCAGAGAGAGCAAGGTATTCTTCAAATACAGACAAGGAAATGATAGTT |
| | TTGAAAATGGTAGATTTGAATGGAGATGACTTGGGCCTTATCAGCTGGTTTGCCATCCACCCGGT |
| | CAGCATGAACAACAGTAACCATCTTGTAAACAGTGACAATGTGGGCTATGCATCTTACCTGCTTG |
| | AGCAAGAGAAGAACAAAGGATATCTACCTGGACAGGGGCCATTTGTAGCAGCCTTTGCTTCATCA |
| | AACCTAGGAGATGTGTCCCCCAACATTCTTGGACCACGTTGCATCAACACAGGAGAGTCCTGTGA |
| | TAACGCCAATAGCACTTGTCCCATTGGTGGGCCTAGCATGTGCATTGCTAAGGGACCTGGACAGG |
| | ATATGTTTGACAGCACACAAATTATAGGACGGGCCATGTATCAGAGAGCAAAGGAACTCTATGCC |
| | TCTGCCTCCCAGGAGGTAACAGGACCACTGGCTTCAGCACACCAGTGGGTGGATATGACAGATG |
| | TGACTGTCTGGCTCAATTCCACACATGCATCAAAAACATGTAAACCAGCATTGGGCTACAGTTTTG |
| | CAGCTGGCACTATTGATGGAGTTGGAGGCCTCAATTTTACACAGGGGAAAACAGAAGGGGATCC |
| | ATTTTGGGACACCATTCGGGACCAGATCCTGGGAAAGCCATCTGAAGAAATTAAAGAATGTCATA |
| | AACCAAAGCCCATCCTTCTTCACACCGGAGAACTATCAAAACCTCACCCCTGGCATCCAGACATTG |
| | TTGATGTTCAGATTATTACCCTTGGGTCCTTGGCCATAACTGCCATCCCCGGGGAGTTTACGACCA |
| | TGTCTGGACGAAGACTTCGAGAGGCAGTTCAAGCAGAATTTGCATCTCATGGGATGCAGAACAT |
| | GACTGTTGTTATTTCAGGTCTATGCAACGTCTATACACATTACATTACCACTTATGAAGAATACCA |
| | GGCTCAGCGATATGAGGCAGCATCGACAATTTATGGACCGCACACATTATCTGCTTACATTCAGC |
| | TCTTCAGAAACCTTGCTAAGGCTATTGCTACGGACACGGTAGCCAACCTGAGCAGAGGTCCAGAA |
| | CCTCCCTTTTTCAAACAATTAATAGTTCCATTAATTCCTAGTATTGTGGATAGAGCACCAAAAGGC |
| | AGAACTTTCGGGGATGTCCTGCAGCCAGCAAAACCTGAATACAGAGTGGGGGAAGTTGCTGAAG |
| | TTATATTTGTAGGTGCTAACCCGAAGAATTCAGTACAAAACCAGACCCATCAGACCTTCCTCACTG |
| | TGGAGAAATATGAGGCTACTTCAACATCGTGGCAGATAGTGTGTAATGATGCCTCCTGGGAGACT |
| | CGTTTTTATTGGCACAAGGGACTCCTGGGTCTGAGTAATGCAACAGTGGAATGGCATATTCCAGA |
| | CACTGCCCAGCCTGGAATCTACAGAATAAGATATTTTGGACACAATCGGAAGCAGGACATTCTGA |
| | AGCCTGCTGTCATACTTTCATTTGAAGGCACTTCCCCGGCTTTTGAAGTTGTAACTATTTAGTGA |
| | (SEQ ID NO: 8) |
| ASAH2 | ATGGCCAAACGCACCTTCTCTAACTTGGAGACATTCCTGATTTTCCTCCTTGTAATGATGAGTGCC |
| transcript | ATCACAGTGGCCCTTCTCAGCCTCTTGTTTATCACCAGTGGGACCATTGAAAACCACAAAGATTTA |
| variant 2 | GGAGGCCATTTTTTTTCAACCACCCAAAGCCCTCCAGCCACCCAGGGCTCCACAGCTGCCCAACGC |
| | TCCACAGCCACCCAGCATTCCACAGCCACCCAGAGCTCCACAGCCACTCAAACTTCTCCAGTGCCT |
| | TTAACCCCAGAGTCTCCTCTATTTCAGAACTTCAGTGGCTACCATATTGGTGTTGGACGAGCTGAC |
| | TGCACAGGACAAGTAGCAGATATCAATTTGATGGGCTATGGCAAATCCGGCCAGAATGCACAGG |
| | GCATCCTCACCAGGCTATACAGTCGTGCCTTCATCATGGCAGAACCTGATGGGTCCAATCGAACA |
| | GTGTTTGTCAGCATCGACATAGGCATGGTATCACAAAGGCTCAGGCTGGAGGTCCTGAACAGAC |
| | TGCAGAGTAAATATGGCTCCCTGTACAGAAGAGATAATGTCATCCTGAGTGGCACTCACACTCAT |
| | TCAGGTCCTGCAGGATATTTCCAGTATACCGTGTTTGTAATTGCCAGTGAAGGATTTAGCAATCAA |
| | ACTTTTCAGCACATGGTCACTGGTATCTTGAAGAGCATTGACATAGCACACACAAATATGAAACC |
| | AGGCAAAATCTTCATCAATAAAGGAAATGTGGATGGTGTGCAGATCAACAGAAGTCCGTATTCTT |
| | ACCTTCAAAATCCGCAGTCAGAGAGAGCAAGGTATTCTTCAAATACAGACAAGGAAATGATAGTT |
| | TTGAAAATGGTAGATTTGAATGGAGATGACTTGGGCCTTATCAGCTGGTTTGCCATCCACCCGGT |
| | CAGCATGAACAACAGTAACCATCTTGTAAACAGTGACAATGTGGGCTATGCATCTTACCTGCTTG |
| | AGCAAGAGAAGAACAAAGGATATCTACCTGGACAGGGGCCATTTGTAGCAGCCTTTGCTTCATCA |
| | AACCTAGGAGATGTGTCCCCCAACATTCTTGGACCACGTTGCATCAACACAGGAGAGTCCTGTGA |
| | TAACGCCAATAGCACTTGTCCCATTGGTGGGCCTAGCATGTGCATTGCTAAGGGACCTGGACAGG |
| | ATATGTTTGACAGCACACAAATTATAGGACGGGCCATGTATCAGAGAGCAAAGTCAAAAACATGT |
| | AAACCAGCATTGGGCTACAGTTTTGCAGCTGGCACTATTGATGGAGTTGGAGGCCTCAATTTTAC |
| | ACAGGGGAAAACAGAAGGGGATCCATTTTGGGACACCATTCGGGACCAGATCCTGGGAAAGCC |
| | ATCTGAAGAAATTAAAGAATGTCATAAACCAAAGCCCATCCTTCTTCACACCGGAGAACTATCAA |
| | AACCTCACCCCTGGCATCCAGACATTGTTGATGTTCAGATTATTACCCTTGGGTCCTTGGCCATAA |
| | CTGCCATCCCCGGGGAGTTTACGACCATGTCTGGACGAAGACTTCGAGAGGCAGTTCAAGCAGA |
| | ATTTGCATCTCATGGGATGCAGAACATGACTGTTGTTATTTCAGGTCTATGCAACGTCTATACACA |
| | TTACATTACCACTTATGAAGAATACCAGGCTCAGCGATATGAGGCAGCATCGACAATTTATGGAC |
| | CGCACACATTATCTGCTTACATTCAGCTCTTCAGAAACCTTGCTAAGGCTATTGCTACGGACACGG |
| | TAGCCAACCTGAGCAGAGGTCCAGAACCTCCCTTTTTCAAACAATTAATAGTTCCATTAATTCCTA |
| | GTATTGTGGATAGAGCACCAAAAGGCAGAACTTTCGGGGATGTCCTGCAGCCAGCAAAACCTGA |
| | ATACAGAGTGGGGGAAGTTGCTGAAGTTATATTTGTAGGTGCTAACCCGAAGAATTCAGTACAA |
| | AACCAGACCCATCAGACCTTCCTCACTGTGGAGAAATATGAGGCTACTTCAACATCGTGGCAGAT |
| | AGTGTGTAATGATGCCTCCTGGGAGACTCGTTTTTATTGGCACAAGGGACTCCTGGGTCTGAGTA |
| | ATGCAACAGTGGAATGGCATATTCCAGACACTGCCCAGCCTGGAATCTACAGAATAAGATATTTT |
| | GGACACAATCGGAAGCAGGACATTCTGAAGCCTGCTGTCATACTTTCATTTGAAGGCACTTCCCC |
| | GGCTTTTGAAGTTGTAACTATTTAGTGA (SEQ ID NO: 9) |
| ASAH2B | ATGAGGCAGCATCGACAATTTATGGACCGCACGCATTATCTGCTTACATTCAGCTCTTCAGAAACC |
| transcript | TTGCTAAGGCTATTGCTACGTATTGTGGATAGAGCACCAAAAGGCAGAACTTTCGGGGATGTCCT |
| variant 1 | GCAGCCAGCAAAACCTGAATACAGAGTGGGGGAAGTTGCTGAAGTTATATTTGTAGGTGCTAAC |
| | CCGAAGAATTCAGTACAAAACCAGACCCATCAGACCTTCCTCACTGTGGAGAAATATGAGGCTAC |
| | TTCAACATCGTGGCAGATAGTGTGTAATGATGCCTCCTGGGAGACTCGTTTTTATTGGCACAAGG |
| | GACTCCTGGGTCTGAGTAATGCAACAGTGGAATGGCATATTCCAGACACTGCCCAGCCTGGAATC |
| | TACAGAATAAGATATTTTGGACACAATCGGAAGCAGGACATTCTGAAGCCTGCTGTCATACTTTC |
| | ATTTGAAGGCACTTCCCCGGCTTTTGAAGTTGTAACTATTTAGTGA (SEQ ID NO: 10) |
| ASAH2B | ATGGTAGCCAACCTGAGCAGAGGTCCAGAACCTCCCTTTTTCAAACAATTAATAGTTCCATTAATT |
| transcript | CCTAGTATTGTGGATAGAGCACCAAAAGGCAGAACTTTCGGGGATGTCCTGCAGCCAGCAAAAC |
| variant 3 | CTGAATACAGAGTGGGGGAAGTTGCTGAAGTTATATTTGTAGGTGCTAACCCGAAGAATTCAGT |
| | ACAAAACCAGACCCATCAGACCTTCCTCACTGTGGAGAAATATGAGGCTACTTCAACATCGTGGC |
| | AGATAGTGTGTAATGATGCCTCCTGGGAGACTCGTTTTTATTGGCACAAGGGACTCCTGGGTCTG |
| | AGTAATGCAACAGTGGAATGGCATATTCCAGACACTGCCCAGCCTGGAATCTACAGAATAAGATA |
| | TTTTGGACACAATCGGAAGCAGGACATTCTGAAGCCTGCTGTCATACTTTCATTTGAAGGCACTTC |
| | CCCGGCTTTTGAAGTTGTAACTATTTAGTGAATGGTAGCCAACCTGAGCAGAGGTCCAGAACCTC |
| | CCTTTTTCAAACAATTAATAGTTCCATTAATTCCTAGTATTGTGGATAGAGCACCAAAAGGCAGAA |
| | CTTTCGGGGATGTCCTGCAGCCAGCAAAACCTGAATACAGAGTGGGGGAAGTTGCTGAAGTTAT |
| | ATTTGTAGGTGCTAACCCGAAGAATTCAGTACAAAACCAGACCCATCAGACCTTCCTCACTGTGG |
| | AGAAATATGAGGCTACTTCAACATCGTGGCAGATAGTGTGTAATGATGCCTCCTGGGAGACTCGT |
| | TTTTATTGGCACAAGGGACTCCTGGGTCTGAGTAATGCAACAGTGGAATGGCATATTCCAGACAC |
| | TGCCCAGCCTGGAATCTACAGAATAAGATATTTTGGACACAATCGGAAGCAGGACATTCTGAAGC |
| | CTGCTGTCATACTTTCATTTGAAGGCACTTCCCCGGCTTTTGAAGTTGTAACTATTTAGTGA |
| | (SEQ ID NO: 11) |
| ASAH2B | ATGGTAGCCAACCTGAGCAGAGGTCCAGAACCTCCCTTTTTCAAACAATTAATAGTTCCATTAATT |
| transcript | CCTAGTATTGTGGATAGAGCACCAAAAGGCAGAACTTTCGGGGATGTCCTGCAGCCAGCAAAAC |
| variant 4 | CTGAATACAGAGTGGGGGAAGTTGCTGAAGTTATATTTGTAGGTGCTAACCCGAAGAATTCAGT |
| | ACAAAACCAGACCCATCAGACCTTCCTCACTGTGGAGAAATATGAGGCTACTTCAACATCGTGGC |
| | AGATAGTGTGTAATGATGCCTCCTGGGAGACTCGTTTTTATTGGCACAAGGGACTCCTGGGTCTG |
| | AGTAATGCAACAGTGGAATGGCATATTCCAGACACTGCCCAGCCTGGAATCTACAGAATAAGATA |
| | TTTTGGACACAATCGGAAGCAGGACATTCTGAAGCCTGCTGTCATACTTTCATTTGAAGGCACTTC |
| | CCCGGCTTTTGAAGTTGTAACTATTTAG (SEQ ID NO: 12) |
| ACER1 | ATGCCTAGCATCTTCGCCTATCAGAGCTCCGAGGTGGACTGGTGTGAGAGCAACTTCCAGTACTC |
| | GGAGCTGGTGGCCGAGTTCTACAACACGTTCTCCAATATCCCCTTCTTCATCTTCGGGCCACTGAT |
| | GATGCTCCTGATGCACCCGTATGCCCAGAAGCGCTCCCGCTACATTTACGTTGTCTGGGTCCTCTT |
| | CATGATCATAGGCCTGTTCTCCATGTATTTCCACATGACGCTCAGCTTCCTGGGCCAGCTGCTGGA |
| | CGAGATCGCCATCCTGTGGCTCCTGGGCAGTGGCTATAGCATATGGATGCCCCGCTGCTATTTCC |
| | CCTCCTTCCTTGGGGGGAACAGGTCCCAGTTCATCCGCCTGGTCTTCATCACCACTGTGGTCAGCA |
| | CCCTTCTGTCCTTCCTGCGGCCCACGGTCAACGCCTACGCCCTCAACAGCATTGCCCTGCACATTCT |
| | CTACATCGTGTGCCAGGAGTACAGGAAGACCAGCAATAAGGAGCTTCGGCACCTGATTGAGGTC |
| | TCCGTGGTTTTATGGGCTGTTGCTCTGACCAGCTGGATCAGTGACCGTCTGCTTTGCAGCTTCTGG |
| | CAGAGGATTCATTTCTTCTATCTGCACAGCATCTGGCATGTGCTCATCAGCATCACCTTCCCTTATG |
| | GCATGGTCACCATGGCCTTGGTGGATGCCAACTATGAGATGCCAGGTGAAACCCTCAAAGTCCGC |
| | TACTGGCCTCGGGACAGTTGGCCCGTGGGGCTGCCCTACGTGGAAATCCGGGGTGATGACAAGG |
| | ACTGCTGA(SEQ ID NO: 13) |
| ACER2 | ATGGGCGCCCCGCACTGGTGGGACCAGCTGCAGGCTGGTAGCTCGGAGGTGGACTGGTGCGAG |
| | GACAACTACACCATCGTGCCTGCTATCGCCGAGTTCTACAACACGATCAGCAATGTCTTATTTTTC |
| | ATTTTACCGCCCATCTGCATGTGCTTGTTTCGTCAGTATGCAACATGCTTCAACAGTGGCATCTACT |
| | TAATCTGGACTCTTTTGGTTGTAGTGGGAATTGGATCCGTCTACTTCCATGCAACCCTTAGTTTCTT |
| | GGGTCAGATGCTTGATGAACTTGCAGTCCTTTGGGTTCTGATGTGTGCTTTGGCCATGTGGTTCCC |
| | CAGAAGGTATCTACCAAAGATCTTTCGGAATGACCGGGGTAGGTTCAAGGTGGTGGTCAGTGTC |
| | CTGTCTGCGGTTACGACGTGCCTGGCATTTGTCAAGCCTGCCATCAACAACATCTCTCTGATGACC |
| | CTGGGAGTTCCTTGCACTGCACTGCTCATCGCAGAGCTAAAGAGGTGTGACAACATGCGTGTGTT |
| | TAAGCTGGGCCTCTTCTCGGGCCTCTGGTGGACCCTGGCCCTGTTCTGCTGGATCAGTGACCGAG |
| | CTTTCTGCGAGCTGCTGTCATCCTTCAACTTCCCCTACCTGCACTGCATGTGGCACATCCTCATCTG |
| | CCTTGCTGCCTACCTGGGCTGTGTATGCTTTGCCTACTTTGATGCTGCCTCAGAGATTCCTGAGCA |
| | AGGCCCTGTCATCAAGTTCTGGCCCAATGAGAAATGGGCCTTCATTGGTGTCCCCTATGTGTCCCT |
| | CCTGTGTGCCAACAAGAAATCATCAGTCAAGATCACGTGA (SEQ ID NO: 14) |
| ACER3 | ATGGCTCCGGCCGCGGACCGAGAGGGCTACTGGGGCCCCACGACCTCCACGCTGGACTGGTGCG |
| transcript | AGGAGAACTACTCCGTGACCTGGTACATCGCCGAGTTCTGGAATACAGTGAGTAACCTGATCATG |
| variant 1 | ATTATACCTCCAATGTTCGGTGCAGTTCAGAGTGTTAGAGACGGTCTGGAAAAGCGGTACATTGC |
| | TTCTTATTTAGCACTCACAGTGGTAGGAATGGGATCCTGGTGCTTCCACATGACTCTGAAATATGA |
| | AATGCAGCTATTGGATGAACTCCCAATGATATACAGCTGTTGCATATTTGTGTACTGCATGTTTGA |
| | ATGTTTCAAGATCAAGAACTCAGTAAACTACCATCTGCTTTTTACCTTAGTTCTATTCAGTTTAATA |
| | GTAACCACAGTTTACCTTAAGGTAAAAGAGCCGATATTCCATCAGGTCATGTATGGAATGTTGGT |
| | CTTTACATTAGTACTTCGATCTATTTATATTGTTACATGGGTTTATCCATGGCTTAGAGGACTGGGT |
| | TATACATCATTGGGTATATTTTTATTGGGATTTTTATTTTGGAATATAGATAACATATTTTGTGAGT |
| | CACTGAGGAACTTTCGAAAGAAGGTACCACCTATCATAGGTATTACCACACAATTTCATGCATGG |
| | TGGCATATTTTAACTGGCCTTGGTTCCTATCTTCACATCCTTTTCAGTTTGTATACAAGAACACTTT |
| | ACCTGAGATATAGGCCAAAAGTGAAGTTTCTCTTTGGAATCTGGCCAGTGATCCTGTTTGAGCCTC |
| | TCAGGAAGCATTGA (SEQ ID NO: 15) |
| ACER3 | ATGGCTCCGGCCGCGGACCGAGAGGGCTACTGGGGCCCCACGACCTCCACGCTGGACTGGTGCG |
| transcript | AGGAGAACTACTCCGTGACCTGGTACATCGCCGAGTTCTTGGTAGGAATGGGATCCTGGTGCTTC |
| variant 2 | CACATGACTCTGAAATATGAAATGCAGCTATTGGATGAACTCCCAATGATATACAGCTGTTGCAT |
| | ATTTGTGTACTGCATGTTTGAATGTTTCAAGATCAAGAACTCAGTAAACTACCATCTGCTTTTTACC |
| | TTAGTTCTATTCAGTTTAATAGTAACCACAGTTTACCTTAAGGTAAAAGAGCCGATATTCCATCAG |
| | GTCATGTATGGAATGTTGGTCTTTACATTAGTACTTCGATCTATTTATATTGTTACATGGGTTTATC |
| | CATGGCTTAGAGGACTGGGTTATACATCATTGGGTATATTTTTATTGGGATTTTTATTTTGGAATA |
| | TAGATAACATATTTTGTGAGTCACTGAGGAACTTTCGAAAGAAGGTACCACCTATCATAGGTATT |
| | ACCACACAATTTCATGCATGGTGGCATATTTTAACTGGCCTTGGTTCCTATCTTCACATCCTTTTCA |
| | GTTTGTATACAAGAACACTTTACCTGAGATATAGGCCAAAAGTGAAGTTTCTCTTTGGAATCTGGC |
| | CAGTGATCCTGTTTGAGCCTCTCAGGAAGCATTGA (SEQ ID NO: 16) |
| ACER3 | ATGATATACAGCTGTTGCATATTTGTGTACTGCATGTTTGAATGTTTCAAGATCAAGAACTCAGTA |
| transcript | AACTACCATCTGCTTTTTACCTTAGTTCTATTCAGTTTAATAGTAACCACAGTTTACCTTAAGGTAA |
| variant 3 | AAGAGCCGATATTCCATCAGGTCATGTATGGAATGTTGGTCTTTACATTAGTACTTCGATCTATTT |
| | ATATTGTTACATGGGTTTATCCATGGCTTAGAGGACTGGGTTATACATCATTGGGTATATTTTTAT |
| | TGGGATTTTTATTTTGGAATATAGATAACATATTTTGTGAGTCACTGAGGAACTTTCGAAAGAAG |
| | GTACCACCTATCATAGGTATTACCACACAATTTCATGCATGGTGGCATATTTTAACTGGCCTTGGT |
| | TCCTATCTTCACATCCTTTTCAGTTTGTATACAAGAACACTTTACCTGAGATATAGGCCAAAAGTGA |
| | AGTTTCTCTTTGGAATCTGGCCAGTGATCCTGTTTGAGCCTCTCAGGAAGCATTGA |
| | (SEQ ID NO: 17) |
| Sphk2 | ATGAATGGACACCTTGAAGCAGAGGAGCAGCAGGACCAGAGGCCAGACCAGGAGCTGACCGGG |
| | AGCTGGGGCCACGGGCCTAGGAGCACCCTGGTCAGGGCTAAGGCCATGGCCCCGCCCCCACCGC |
| | CACTGGCTGCCAGCACCCCGCTCCTCCATGGCGAGTTTGGCTCCTACCCAGCCCGAGGCCCACGC |
| | TTTGCCCTCACCCTTACATCGCAGGCCCTGCACATACAGCGGCTGCGCCCCAAACCTGAAGCCAG |
| | GCCCCGGGGTGGCCTGGTCCCGTTGGCCGAGGTCTCAGGCTGCTGCACCCTGCGAAGCCGCAGC |
| | CCCTCAGACTCAGCGGCCTACTTCTGCATCTACACCTACCCTCGGGGCCGGCGCGGGGCCCGGCG |
| | CAGAGCCACTCGCACCTTCCGGGCAGATGGGGCCGCCACCTACGAAGAGAACCGTGCCGAGGCC |
| | CAGCGCTGGGCCACTGCCCTCACCTGTCTGCTCCGAGGACTGCCACTGCCCGGGGATGGGGAGA |
| | TCACCCCTGACCTGCTACCTCGGCCGCCCCGGTTGCTTCTATTGGTCAATCCCTTTGGGGGTCGGG |
| | GCCTGGCCTGGCAGTGGTGTAAGAACCACGTGCTTCCCATGATCTCTGAAGCTGGGCTGTCCTTC |
| | AACCTCATCCAGACAGAACGACAGAACCACGCCCGGGAGCTGGTCCAGGGGCTGAGCCTGAGTG |
| | AGTGGGATGGCATCGTCACGGTCTCGGGAGACGGGCTGCTCCATGAGGTGCTGAACGGGCTCCT |
| | AGATCGCCCTGACTGGGAGGAAGCTGTGAAGATGCCTGTGGGCATCCTCCCCTGCGGCTCGGGC |
| | AACGCGCTGGCCGGAGCAGTGAACCAGCACGGGGGATTTGAGCCAGCCCTGGGCCTCGACCTGT |
| | TGCTCAACTGCTCACTGTTGCTGTGCCGGGGTGGTGGCCACCCACTGGACCTGCTCTCCGTGACG |
| | CTGGCCTCGGGCTCCCGCTGTTTCTCCTTCCTGTCTGTGGCCTGGGGCTTCGTGTCAGATGTGGAT |
| | ATCCAGAGCGAGCGCTTCAGGGCCTTGGGCAGTGCCCGCTTCACACTGGGCACGGTGCTGGGCC |
| | TCGCCACACTGCACACCTACCGCGGACGCCTCTCCTACCTCCCCGCCACTGTGGAACCTGCCTCGC |
| | CCACCCCTGCCCATAGCCTGCCTCGTGCCAAGTCGGAGCTGACCCTAACCCCAGACCCAGCCCCG |
| | CCCATGGCCCACTCACCCCTGCATCGTTCTGTGTCTGACCTGCCTCTTCCCCTGCCCCAGCCTGCCC |
| | TGGCCTCTCCTGGCTCGCCAGAACCCCTGCCCATCCTGTCCCTCAACGGTGGGGGCCCAGAGCTG |
| | GCTGGGGACTGGGGTGGGGCTGGGGATGCTCCGCTGTCCCCGGACCCACTGCTGTCTTCACCTC |
| | CTGGCTCTCCCAAGGCAGCTCTACACTCACCCGTCTCCGAAGGGGCCCCCGTAATTCCCCCATCCT |
| | CTGGGCTCCCACTTCCCACCCCTGATGCCCGGGTAGGGGCCTCCACCTGCGGCCCGCCCGACCAC |
| | CTGCTGCCTCCGCTGGGCACCCCGCTGCCCCCAGACTGGGTGACGCTGGAGGGGGACTTTGTGC |
| | TCATGTTGGCCATCTCGCCCAGCCACCTAGGCGCTGACCTGGTGGCAGCTCCGCATGCGCGCTTC |
| | GACGACGGCCTGGTGCACCTGTGCTGGGTGCGTAGCGGCATCTCGCGGGCTGCGCTGCTGCGCC |
| | TTTTCTTGGCCATGGAGCGTGGTAGCCACTTCAGCCTGGGCTGTCCGCAGCTGGGCTACGCCGCG |
| | GCCCGTGCCTTCCGCCTAGAGCCGCTCACACCACGCGGCGTGCTCACAGTGGACGGGGAGCAGG |
| | TGGAGTATGGGCCGCTACAGGCACAGATGCACCCTGGCATCGGTACACTGCTCACTGGGCCTCCT |
| | GGCTGCCCGGGGCGGGAGCCCTGA (SEQ ID NO: 18) |

### Modes of Delivery

Modified mRNA (modRNA)

modRNA is a synthetic mRNA with an optimized 5′UTR and 3′UTR sequences, anti-reverse cup analog (ARCA) and one or more naturally modified nucleotides. The optimized UTRs sequences enhance the translation efficiency. ARCA increases the stability of the RNA and enhances the translation efficiency and the naturally modified nucleotides increase the stability of the RNA reduce the innate immune response of cells (in vitro and in vivo) and enhance the translation efficiency of the mRNA. This combination generates a superior mRNA that mediate a higher and longer expression of proteins with a minimal immune respond. Modified mRNA is a safe, local, transient, and with high expression gene delivery method to the heart.

Kariko et al. have shown that uridine replacement in mRNA with pseudouridine (hence the name modified mRNA (modRNA)) resulted in changes to the mRNA secondary structure that avoid the innate immune system and reduce the recognition of modRNA by RNase. In addition, these changes of nucleotides are naturally occurring in our body and lead to enhanced translation of the modRNA compared to unmodified mRNA.

Accordingly, advantages of using modRNA are as follows. One advantage of modRNA delivery is the lack of a requirement for nuclear localization or transcription prior to translation of the gene of interest. Eliminating the need for transcription of an mRNA prior to translation of the protein of interest results in higher efficiency in expression of the protein of interest.

Another advantage is the nearly negligible possibility of genomic integration of the delivered sequence. The use of viruses or DNA to facilitate expression of a protein of interest permanently alters the genome of the cells and can introduce risk that the vector will inadvertently cause the expression of other, undesirable genes.

Thirdly, messenger RNA modifications allow modRNA to avoid detection by the innate immune system and RNase. Based on that observation, modRNA can be used as a safe and effective tool for scenarios in which short-term gene delivery is desired. Pharmacokinetics analyses of modRNA indicate a pulse-like expression of protein up to 7 days. The use of modRNA, a relatively nascent technology, has considerable potential as a therapy for disease. Delivery of a synthetic modified RNA encoding human vascular endothelial growth factor-A, for example, results in expansion and directed differentiation of endogenous heart progenitors in a mouse myocardial infarction model (Zangi et al. Modified mRNA directs the fate of heart progenitor cells and induces vascular regeneration after myocardial infarction. Nature Biotechnology 31, 898-907 (2013)). Diabetic neuropathy may be lessened by the ability to deliver genes encoding nerve growth factor. Additionally, with the advent of genome editing technology, CRISPR/Cas9 or transcription activator-like effector nuclease (TALEN), transfection will be safer if delivered in a transient and cell-specific manner.

In one embodiment of the present method, the nucleic acid that encodes a sphingolipid-metabolizing protein is modRNA. While various gene delivery methods are suitable for achieving expression of an exogenous protein, for example, plasmids and viruses, for certain indications, modRNA offers several advantages as a gene delivery tool. For example, the use of mRNA as a gene delivery method to mammalian tissue has been very limited. This is mostly due to the immunogenicity of mRNA, via activation of Toll like receptors 7/8 or 3. In addition, mRNA is prone to cleavage by RNase in the blood when delivered in vivo.

Since the modRNAs encode physiological enzymes, the expression of ceramidase should have little or no toxic effects. In addition, transfecting cells with ceramidase modRNA will increase the precursor (inactive form) of the enzyme that will allow autonomous control of the active ceramidase protein, which is required for survival. Furthermore, control of ceramide metabolism is the only known biological function of ceramidase; manipulation of ceramidase should not influence other cellular signaling. In addition, creation of a mouse model that continually overexpresses the AC enzyme (COEAC) in all tissues demonstrates a lack of toxicity or tumorigenesis effect by overexpression of AC.

### Technologies

Nowhere is the role of apoptosis more significant than in the field of reproduction. Ovulated oocytes undergo molecular changes characteristic of apoptosis unless successful fertilization occurs. Under normal physiological conditions 85-90% of oocytes succumb to apoptosis at some point during fetal or postnatal life. Clinically, when the remaining oocyte reserve has been exhausted (on average, this occurs in women around age 50), menopause ensues as a direct consequence of ovarian senescence. A major challenge of assisted reproduction technologies (ARTs) is to mimic the natural environment required to sustain oocyte and embryo survival.

Accordingly, the ability to increase cell quality and survival is of particular interest in reproductive cells, which have unique features, such as the ability of the oocyte to undergo a cortical reaction and triggering of protein expression in the fertilized zygote.

The formation of a human embryo starts with the fertilization of the oocyte by the sperm cell. This yields the zygote, which carries one copy each of the maternal and paternal genomes. To prevent fertilization by multiple sperm, the egg undergoes a cortical reaction; once a single sperm manages to penetrate the outer membrane of the oocyte, the oocyte develops a permanent, impermeable barrier.

Expression of the genetic information contained in the zygote starts only after the zygote divides a couple of times.

There are several studies that support association of the signaling lipid, ceramide, and its metabolizing enzymes with cellular and organismal aging. It has been reported that the intracellular level of ceramide increased during stress related signaling such as cell culture and aging. Ceramidase, for example, acid ceramidase (AC) is required to hydrolyze ceramide into sphingosine and free fatty acids. Sphingosine is rapidly converted to sphingosine-1-phosphate (S1P), another important signaling lipid that counteracts the effects of ceramide and promotes cell survival. Thus, AC is a “rheostat” that regulates the levels of ceramide and S1P in cells, and as such participates in the complex and delicate balance between death and survival.

We have previously shown that AC expression is carefully regulated during oocyte maturation and early embryo development (Eliyahu, et al, 2010). We have also found that the complete “knock-out” of AC function in mice leads to embryo death between the 2 and 8-cell stage (Eliyahu, FASEB J, 2007). In addition, our previous publication (Eliyahu, FASEB J, 2010) showed that the ceramide-metabolizing enzyme, AC is expressed and active in human cumulus cells and follicular fluid, essential components of this environment, and that the levels of this enzyme are positively correlated with the quality of human embryos formed in vitro. These observations led to a new approach for oocyte and embryo culture that markedly improves the outcome of in vitro fertilization (IVF).

The disclosed method provides an opportunity to improve egg quality. Egg quality is important for successful fertility treatment. Couples who have a failed IVF cycle, or are considering undergoing IVF at an advanced maternal age, are often told that they have poor-quality eggs. The simple fact is that high-quality eggs produce high-quality embryos. Embryos must be healthy and robust enough to survive the early stages of development in order to result in a successful pregnancy.

As a woman ages, her ovaries' ability to retain high-quality eggs starts to decline. This is a condition known as diminished ovarian reserve (DOR) and is the most common cause of infertility for women over 40. These women have difficulty conceiving, to a large extent because of the poor egg quality resulting in poor embryo quality. Success rates of fertility treatments are also lower for these couples, who are often referred for adoption or donor egg. The method disclosed herein provides a treatment plan to improve the quality of eggs and embryos.

### Expression of Sphingolipid-Metabolizing Proteins to Improve the Efficiency of Gene Editing

The CRISPR System

The CRISPR with Cas 9 system (CRISPR-Cas9 system) is now well known in the art as a gene-editing platform. CRISPR-Cas9 is an adaptive immune defense mechanism used by Archea and bacteria for the degradation of foreign genetic elements and uses specially designed RNAs that guide the Cas9 nuclease to the target DNA where it induces genomic engineering for mammalian systems, such as gene knockout DNA breaks.

These breaks are repaired either by non-homologous end-joining (NHEJ), which is an error-prone process that leads to other insertions or deletions, or by homology-directed repair (HDR), which requires a template but is less error prone. CRISPR-Cas9 system for site-specific genome engineering open the possibility to perform rapid targeted genome modification in virtually any laboratory species without the need to rely on embryonic stem (ES) cell technology. Impressively, by directly injecting Cas9 mRNA and single-guide RNAs (sgRNAs) into zygotes, mice or rats carrying mutations in transgenes or multiple endogenous genes can be generated in one step (Yang et al., 2013), indicating that the CRISPR-Cas9 system can be used as an effective tool for genome engineering.

The CRISPR-Cas9 system has been employed to generate mutant alleles in a range of different organisms, including C. elegans, zebrafish, mouse, rat, monkey and human. Recently, the CRISPR-Cas9 method of genetic recombination was used to correct genetic defects in human adult stem cell organoids, and in mouse zygotes for the first time. The CRISPR-Cas9 system was used to correct mice with a dominant mutation in the Crygc gene that causes cataracts.

More recently, researchers demonstrated successful gene editing in human embryos (Ruzo and Brivanlou, 2017). These studies suggest that the CRISPR-Cas system could be used for human gene therapy to correct genetic defect not only in affected patient but also in their germ line, which ensure that their progeny won't be affected.

Effect of Acid Ceramidase on Gene Editing with CRISPR

The methodology disclosed herein improves the Preimplantation Genetic Editing (PGE) technology by: (1) Improving embryo vitality and survival rate post CRISPR cocktail injection; (2) improving DNA repair in order to reduce off target deletions/insertion; (3) improving the efficiency of CRISPR technology (positive pups for DNA editing) in order to extend the mutation correction/creation to wide research and clinical use, including genetic diseases or other diseases such as cancer in animal and human.

Our results demonstrate dramatic improvement of embryo survival, higher number of live born pups and higher successful gene editing rate post CRISPR injection (Table 2-5).

These observations led to a new approach for oocyte and embryo culture that markedly improves efficiency of Clustered regularly-interspaced short palindromic repeats (CRISPR) technology when applied to these cells and others.

Our results demonstrate dramatic improvement of embryo survival post CRISPR injection at 2 h post injection, 2-4 cell stage and at blastocysts stage (Table 2 and 3).

TABLE 2

| | Injected | Survival 2 h | 2-4 cells |
|---|---|---|---|
| Conditions | Zygotes | Post Injection | embryos |
| CRISPR ONLY | 322 | 164/322 (76%) | 139/322 (43%) |
| CRISPR + | 316 | 295/316 (93%)* | 247/316 (78%)** |
| AC ModRNA | | | |

As shown in Table 2, AC ModRNA improves post CRISPR embryo survival rate. PN embryos were injected with CRISPR cocktail with and without 100 ng of AC modRNA. Embryos were incubated for 2 days in 37° C. CO₂ incubator. Post incubation, embryos were validated for survival. *(P<0.003), **(P<0.003).

TABLE 3

| Conditions | Injected Zygotes | Blastocysts |
|---|---|---|
| CRISPR ONLY | 151 | 27/151 (18%) |
| CRISPR + | 116 | 82/116 (71%)* |
| AC ModRNA | | |

As shown in Table 3, AC ModRNA improves blastocyst survival and quality post CRISPR injection. PN embryos were injected with CRISPR cocktail with and without 100 ng of AC modRNA. Embryos were incubated for 5 days in 37° C. CO₂ incubator. Post incubation, embryos were validated for blastocysts grade. *(P<0.005).

TABLE 4

| Conditions | Injected Zygotes | Pups |
|---|---|---|
| CRISPR ONLY | 107 | 8/107 (7%) |
| CRISPR + | 116 | 19/116 (16%)* |
| AC ModRNA | | |

As shown in Table 4, AC ModRNA improves CRISPR efficiency rate. PN embryos were injected with CRISPR cocktail with and without 100 ng of AC modRNA. Embryos were transferred to host female and numbers of live born recorded. In addition, the results show higher number of live born. *(P<0.003).

Moreover, adjusting the CRISPR microinjection protocol conditions combined with the disclosed survival modRNA factor treatment revealed remarkable improvement of CRISPR efficiency from 15% to 77% (Table 5).

TABLE 5

| Conditions | Successful CRISPR insertion |
|---|---|
| CRISPR ONLY | 15% |
| CRISPR + | 77% |
| AC ModRNA | |

As shown in Table 5, AC ModRNA improves CRISPR efficiency. PN embryos were injected with CRISPR cocktail with and without 100 ng of AC modRNA. Embryos were transferred to host female and number of CRISPR positive life born recorded. *(P<0.003).

### Examples

### Mice

All animal procedures were performed under protocols approved by the Icahn School of Medicine at Mount Sinai or the New York University Transgenic Mouse Facility Institutional Care and Use Committees.

Synthesis of modRNA

Clean PCR products generated with plasmid templates served as template for mRNA. ModRNAs were transcribed in vitro using a custom ribonucleoside blend of Anti Reverse Cap Analog, 3′-O-Me-m7G(5′) ppp(5′)G (6 mM, TriLink Biotechnologies), guanosine triphosphate (1.5 mM, Life Technologies), adenosine triphosphate (7.5 mM, Life Technologies), cytidine triphosphate (7.5 mM, Life Technologies), N1-Methylpseu-douridine-5′-Triphosphate (7.5 mM, TriLink Biotechnologies). The mRNA was purified using a Megaclear kit (Life Technologies) and was treated with Antarctic Phosphatase (New England Biolabs); then it was purified again using the Megaclear kit. The mRNA was quantitated by Nanodrop (Thermo Scientific), precipitated with ethanol and ammonium acetate, and resuspended in 10 mM TrisHCl and 1 mM EDTA.

In Vitro Transfection of modRNA in Sperm and Oocytes

Mouse sperm and oocytes were transfected by adding from 50 to 200 ng/microliter of naked AC modRNA into the culture media. In some embodiments, 100 ng/μl was used. Pronuclei (PN) embryos can be injected with modRNA by intracytoplasmic injection. In some embodiments, embryos were injected with 50-100 ng of modRNA.

Importantly, oocytes and sperm, in contrast to any other cells we tested in vitro were able to be transfected with naked modRNA, that is, no transfection reagent was required. This unique ability will enable the use of modRNA during IVF with no injection.

Real-Time qPCR Analyses

Total RNA was isolated using the RNeasy mini kit (QIAGEN) and reverse transcribed using Superscript III reverse transcriptase (Invitrogen), according to the manufacturer's instructions. Real-time qPCR analyses were performed on a Mastercycler realplex 4 Sequence Detector (Eppendoff) using SYBR Green (Quantitect™ SYBR Green PCR Kit, QIAGEN). Data were normalized to 18srRNA expression where appropriate (endogenous controls). Fold changes of gene expression were determined by the ddCT method. PCR primer sequences are summarized in Table 7.

TABLE 7

| | | SEQ | | SEQ |
|---|---|---|---|---|
| | | ID | | ID |
| Gene | Forward | NO. | Reverse | NO. |
| AC | ACAGGATTCAAACCAGGACTGT | 19 | TGGGCATCTTTCCTTCCGAA | 20 |
| AC | TGACAGGATTCAAACCAGGACT | 21 | CTGGGCATCTTTCCTTCCGA | 22 |
| Sphk1 | ATACTCACCGAACGGAAGAACC | 23 | CCATTAGCCCATTCACCACCTC | 24 |
| Sphk1 | ACTGATACTCACCGAACGGAA | 25 | CATTAGCCCATTCACCACCTC | 26 |
| S1PR2 | CACAGCCAACAGTCTCCAAA | 27 | TCTGAGTATAAGCCGCCCA | 28 |
| S1PR2 | ATAGACCGAGCACAGCCAA | 29 | GAACCTTCTCAGGATTGAGGT | 30 |
| 18s rRNA* | TAACGAACGAGACTCTGGCAT | 31 | CGGACATCTAAGGGCATCACAG | 32 |

*Genetic Vaccines and Therapy 2004, 2:5

### Methods for Assisted Reproduction Studies

Mouse Oocyte and Sperm Collection

All experiments involving animals were approved by and performed in strict accordance with the guidelines of the appropriate institutional animal care and use committees. Seven- to 8-wk-old 129-SVIMJ and C57-Black/6 female mice (Jackson Laboratory, Bar Harbor, Me.) were superovulated with 10 IU of pregnant mare serum gonadotropin (PMSG; Syncro-part, Sanofi, France), followed by 10 IU of human chorionic gonadotropin (hCG; Sigma, St. Louis, Mo.) 48 hours later. Mature and aged MII oocytes were collected from the oviduct ampullae at 16 or 46 hour after injection of hCG, respectively. Cumulus cells were removed by a brief exposure to 400 IU/ml of highly purified hyaluronidase (H-3631; Sigma) in M2 medium (Sigma). Epididymal sperm from 10-wk-old mice were used for IVF of oocytes from the same strain.

### Mouse Fertilization and Embryo Culture

Microdrops of fertile sperm in Vitrofert solution (Vitrolife, Goteborg, Sweden) were prepared, and ˜10 oocytes were placed into each sperm microdrop. The fertilization process was performed for 6 hours at 37° C. in a humidified atmosphere of 5% CO₂ and 95% air. After IVF, zygotes were washed 3 times with potassium simplex optimized medium (KSOM, Chem icon, Billerica Mass.) and cultured for an additional 20-48 hours at 37° C. in a humidified atmosphere of 5% CO₂ and 95% air. Cleavage of the zygotes was observed and recorded throughout the in vitro culture.

### Harvest, Evaluation and Culture of Human Gametes

(A) Oocytes

Female patients undergo approved and controlled ovarian stimulation by administration of recombinant follicle-stimulating hormone (rFSH) followed by concomitant administration of gonadotropin-releasing hormone (GnRH) antagonist. Specifically, rFSH is administrated beginning from a day equal to ½ of the cycle. GnRH antagonist is added at day 6, or when follicles are 12 mm in diameter and until the leading follicle exceeds mm or the estradiol level is above 450 pg/ml. This protocol is continued until at least 2 follicles of 17-18 mm are observed. At this point, ovulation is induced by double trigger administration of Ovitrelle (LH) and Decapeptide (GnRH analogue). Ovum pickup are performed 36-38 h afterwards.

The cumulus-oocyte complexes are isolated into fertilization medium (LifeGlobal), in the presence of 100 μg/μl of AC modRNA.

(B) Sperm

Sperm samples are evaluated for their count, motility and morphology, and all parameters are documented. Post validation sperm are incubated with Multipurpose Handling Medium® (MHM®, Irvine Scientific), and divided into two halves; one half is incubated in the presence of 100 μg/μl of AC modRNA in the media for 1 hour as the study group, and the second half is incubated in the absence of AC modRNA in the media for control. After a 1 hour incubation, a second evaluation of sperm samples for their count, motility and morphology is conducted. Values are compared to those obtained before treatment with AC modRNA.

Following incubation and evaluation, gametes are handled by an approved and common protocol. Oocytes are inseminated, or injected, by ICSI (intracytoplamic sperm injection) according to the spouse sperm parameters and routine protocol. After insemination, ICSI oocytes are transferred to Global medium (medium for culture of Life Global) as is routine in IVF/ICSI. All embryos are incubated and embryonic development is monitored from the time of fertilization up to day 5 in the integrated EmbryoScope™ time-lapse monitoring system (EMBRYOSCOPE™, UnisenseFertiliTech, Vitrolyfe Denmark). The EMBRYOSCOPE™ offers the possibility of continuous monitoring of embryo development without disturbing culture conditions. Embryo scoring and selection with time-lapse monitoring is performed by analysis of time-lapse images of each embryo with software developed specifically for image analysis (EmbryoViewer workstation; UnisenseFertilitech A/S). Embryo morphology and developmental events are recorded to demonstrate the precise timing of the observed cell divisions in correlation to the timing of fertilization as follows: time of 1) pronuclei fading (tPnf), 2) cleavage to a 2-blastomere (t2), 3) 3-blastomere (t3), 4) 4-blastomere (t4) and so forth until reaching an 8-blastomere (t8) embryo, 5) compaction (t_{M}), and 6) start of blastulation. In addition, the synchrony and the duration of cleavages are also measured. Blastocyst morphology including the composition of the inner cell mass and the trophectoderm, are evaluated according to the Gardner blastocyst grading scale.

The addition of recombinant AC (rAC) to young or aged human and mouse oocyte culture medium maintained their healthy morphology in vitro (Eliyahu et al., Acid ceramidase improves the quality of oocytes and embryos and the outcome of in vitro fertilization. FASEB J. 24(4): 1229-1238 2010, the contents of which are incorporated by reference into the present disclosure).

Preimplantation genetic screening (PGS) is performed by chromosomal microarray analysis (CMA) in order to select euploid embryos for transfer. For this, trophectoderm biopsy is performed on day 5. Subsequently, blastocysts and the biopsied embryos are frozen by vitrification. DNA from trophectodermal samples is subjected to whole genome amplification (WGA) and CMA as previously described (Frumkin et al., 2017). Embryos found to be euploid are thawed in a subsequent cycle and transferred to the uterus of the mother for implantation and pregnancy.

Following fertilization, the number of mouse and bovine embryos formed in the presence of AC also was improved (from approximately 40 to 88%), leading to approximately 5-fold more healthy births. Significantly more high-grade blastocysts were formed, and the number of morphologically intact, hatched embryos was increased from approximately 24 to 70% (Eliyahu et al., 2010).

During an IVF protocol embryo culture can last up to 7 days and the chance of embryo survival are low especially for early embryos produced by aged oocytes. As shown in Table 3 mouse oocytes aged in vitro (that serve as a model for oocyte of elderly woman's) have higher chances to develop in to healthy embryos post AC treatment (Fertilization rate increased from 0.02% to 25.2%)(Eliyahu et al., 2010). Since the embryo's gene activation machinery is not fully functional yet, it's very challenging for the embryos to survive for so long in culture.

As part of our effort to prolong embryo survival in culture we developed a method for preventing the apoptotic death of embryos cultured in vitro by administering an effective amount of the Sphingolipids metabolize AC Modified mRNA (modRNA). The present disclosure describes using modRNA rather than recombinant protein based on the observation that modRNA can supply enzyme expression for at least 10 days even post embryo transfer and implantation. Usually during human IVF protocol embryos will be transferred between days 3-5 and it is not possible to expose the embryo post transfer to the recombinant protein. In addition, all embryos will be incubated from the time of fertilization up to day 5 in the integrated EmbryoScope™ time-lapse monitoring system (EmbryoScope™, UnisenseFertiliTech, Vitrolyfe Denmark). The EmbryoScope™ offers the possibility of continuous monitoring of embryo development without disturbing culture conditions. The use of recombinant protein requires disruption of culture condition in order to refresh the media every 24-48 h. (see preliminary results showing S1PR/AC/GFP modRNA's expression in PN embryos (day 1) up to late blastocysts stage (day 7) (FIGS. 2A-2D).

Preliminary results demonstrated that modRNA survival cocktail injection into early mouse embryos dramatically improves the number of formed blastocytes (Table 4) and the number of live-born pups during IVF and embryo injection (Table 5).

Survival effect of AC modRNA was evaluated on the basis of 1) sperm motility, 2) embryo morphology and morphokinetics from day 1-5, 3) blastocyst ploidy, and 4) pregnancy rate.

### CRISPR in Embryos: Microinjection Protocol

Female mice were used to obtain fertilized eggs post superovulation. Fertilized eggs were injected with AC modRNA and CRISPR cocktail reagent following the sgRNA hybridization protocol for zygotes microinjection in accordance with methods known to those of skill in the art.

The control mix contained sgRNA:tracRNA, Cas9 mRNA, HDR Temp, and TE. The AC mix contained Cas9 mRNA, sgRNA, donor DNA and ACmodRNA (at different concentrations).

### REFERENCES

Perez G I, Tao X J, Tilly J L. Fragmentation and death (a.k.a. apoptosis) of ovulated oocytes. Mol Hum Reprod. 1999; 5(5):414-20.
Eliyahu E, Park J H, Shtraizent N, He X, Schuchman E H. Acid ceramidase is a novel factor required for early embryo survival. FASEB J. 2007; 21(7):1403-9.
Eliyahu E, Shtraizent N, Martinuzzi K, Barritt J, He X, Wei H, Chaubal S, Copperman A B, Schuchman E H. Acid ceramidase improves the quality of oocytes and embryos and the outcome of in vitro fertilization. FASEB J. 2010; 24(4):1229-38.
Katalin Karikó, Hiromi Muramatsu, Frank A Welsh, János Ludwig, Hiroki Kato, Shizuo Akira, Drew Weissman. Incorporation of Pseudouridine Into mRNA Yields Superior Nonimmunogenic Vector With Increased Translational Capacity and Biological Stability. Mol Ther. 2008; 16(11): 1833-1840.
Yang H, Wang H, Shivalila C S, Cheng A W, Shi L, Jaenisch R. One-step generation of mice carrying reporter and conditional alleles by CRISPR/Cas-mediated genome engineering. Cell. 2013; 154(6):1370-9.
Wu Y, Liang D, Wang Y, Bai M, Tang W, Bao S, Yan Z, Li D, Li J. Correction of a genetic disease in mouse via use of CRISPR-Cas9. Cell Stem Cell. 2013; 13(6):659-62.
Ruzo A, Brivanlou A H. At Last: Gene Editing in Human Embryos to Understand Human Development. Cell Stem Cell. 2017; 21(5):564-565.
Frumkin T, Peleg S, Gold V, Reches A, Asaf S, Azem F, Ben-Yosef D, Malcov M. Complex chromosomal rearrangement—a lesson learned from PGS. J Assist Reprod Genet. 2017; 34(8):1095-1100.

1. A method for improving the efficiency of gene/genome editing in a mammalian cell or group of mammalian cells, the method comprising:
a. contacting said cell or group of cells undergoing gene editing with a sphingolipid-metabolizing protein or a modRNA that encodes a sphingolipid-metabolizing protein before or during gene editing; and
b. performing gene/genome editing.

2. The method of claim 1, wherein said cells or group of cells are contacted with the sphingolipid-metabolizing protein or modRNA that encodes a sphingolipid-metabolizing protein before gene/genome editing.

3. The method of claim 1, wherein said cells or group of cells are contacted with the sphingolipid-metabolizing protein or modRNA that encodes a sphingolipid-metabolizing protein for a time sufficient for cell uptake of the sphingolipid-metabolizing protein or modRNA that encodes a sphingolipid-metabolizing protein.

4. The method of claim 1, wherein said sphingolipid-metabolizing protein is selected from the group consisting of ceramidase, sphingosine kinase (SPHK), and sphingosine-1-phosphate receptor (S1PR).

5. The method of claim 4, wherein said sphingolipid-metabolizing protein is acid ceramidase (AC).

6. The method of claim 5, wherein the acid ceramidase is encoded by the nucleotide sequence of SEQ ID NO: 1 or SEQ ID NO: 7.

7. The method of claim 4, wherein said sphingolipid-metabolizing protein is neutral ceramidase.

8. The method of claim 7, wherein the neutral ceramidase is encoded by the nucleotide sequence of SEQ ID NO: 6, SEQ ID NO: 8, SEQ ID NO: 9, SEQ ID NO: 10, SEQ ID NO: 11, or SEQ ID NO: 12.

9. The method of claim 4, wherein said sphingolipid-metabolizing protein is alkaline ceramidase.

10. The method of claim 9, wherein the alkaline ceramidase is encoded by the nucleotide sequence of SEQ ID NO: 13, SEQ ID NO: 14, SEQ ID NO: 15, SEQ ID NO: 16 or SEQ ID NO: 17.

11. The method of claim 4, wherein the sphingolipid-metabolizing protein is sphingosine kinase (SPHK).

12. The method of claim 11, wherein SPHK is encoded by the nucleotide sequence of SEQ ID NO: 2.

13. The method of claim 1, wherein said sphingolipid-metabolizing protein is S1PR2.

14. The method of claim 13, wherein the S1PR2 is encoded by the nucleotide sequence of SEQ ID NO: 3.

15. The method of claim 1, wherein the gene/genome editing is performed using a Clustered Regularly Interspaced Short Palindromic Repeats (CRISPR) system.

16. The method of claim 1, wherein said cells or group of cells are contacted with modRNA that encodes ceramidase and modRNA that encodes sphingosine kinase (SPHK).

17. The method of claim 1, wherein said cells or group of cells are contacted with modRNA that encodes ceramidase, modRNA that encodes sphingosine kinase (SPHK) and modRNA that encodes sphingosine-1-phosphate receptor (S1PR).

18. The method of claim 1, wherein said cells or group of cells are contacted with modRNA that encodes ceramidase and modRNA that encodes sphingosine-1-phosphate receptor (S1PR).

19. The method of claim 1, wherein said cells or group of cells are contacted with modRNA that encodes sphingosine kinase (SPHK) and modRNA that encodes sphingosine-1-phosphate receptor (S1PR).

20. The method of claim 1, wherein said mammalian cell or group of cells is selected from the group consisting of primary cells, oocytes, sperm, embryos, and stem cells.

21. The method of claim 1, wherein said cells are cardiac cells.

22. The method of claim 21, wherein said cells are contacted in vivo.


SEQUENCE LISTING (32 sequences)

SEQ ID NO: 1 - DNA, 1188 bp, Artificial Sequence
  synthetic oligonucleotide
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 2 - DNA, 1194 bp, artificial sequence
  synthetic nucleotide
atccagtggtcggttgcggacgtggcctctttggttttgttttctcagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcctacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttatatg

SEQ ID NO: 3 - DNA, 1062 bp, Artificial sequence
  synthetic nucleotide
atgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggaggccgggggtgggggtgcaaggacggaggcggggcgggaccccgggccaccacctcctgccactccgcagctccagctccctggagaggggcatgcacatgcccacgtcacccacgtttctggagggcaacacggtggtcatg

SEQ ID NO: 4 - DNA, 1653 bp, Artificial Sequence
  synthetic nucleotide
atggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaa

SEQ ID NO: 5 - DNA, 801 bp, Artificial Sequence
  synthetic nucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggagatccaaaaaagaagagaaaggtaggcgatccaaaaaagaagagaaaggtaggtgatccaaaaaagaagagaaaggtataa

SEQ ID NO: 6 - DNA, 1236 bp, Artificial Sequence
  synthetic nucleotide
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 7 - DNA, 1170 bp, Artificial Sequence
  synthetic nucleotide
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaactgtcttccctgctgttataaggtacagaggtgcagttccatggtacaccataaatcttgacttaccaccctacaaaagatggcatgaattgatgcttgacaaggcaccagtgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 8 - DNA, 2346 bp, Artificial Sequence
  synthetic nucleotide
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaaggaactctatgcctctgcctcccaggaggtaacaggaccactggcttcagcacaccagtgggtggatatgacagatgtgactgtctggctcaattccacacatgcatcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 9 - DNA, 2241 bp, Artificial Sequence
  synthetic nucleotide
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaagtcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 10 - DNA, 501 bp, Artificial Sequence
  synthetic nucleotide
atgaggcagcatcgacaatttatggaccgcacgcattatctgcttacattcagctcttcagaaaccttgctaaggctattgctacgtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 11 - DNA, 972 bp, Artificial Sequence
  synthetic nucleotide
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtgaatggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 12 - DNA, 483 bp, Artificial Sequence
  synthetic nucleotide
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttag

SEQ ID NO: 13 - DNA, 795 bp, Artificial Sequence
  synthetic nucleotide
atgcctagcatcttcgcctatcagagctccgaggtggactggtgtgagagcaacttccagtactcggagctggtggccgagttctacaacacgttctccaatatccccttcttcatcttcgggccactgatgatgctcctgatgcacccgtatgcccagaagcgctcccgctacatttacgttgtctgggtcctcttcatgatcataggcctgttctccatgtatttccacatgacgctcagcttcctgggccagctgctggacgagatcgccatcctgtggctcctgggcagtggctatagcatatggatgccccgctgctatttcccctccttccttggggggaacaggtcccagttcatccgcctggtcttcatcaccactgtggtcagcacccttctgtccttcctgcggcccacggtcaacgcctacgccctcaacagcattgccctgcacattctctacatcgtgtgccaggagtacaggaagaccagcaataaggagcttcggcacctgattgaggtctccgtggttttatgggctgttgctctgaccagctggatcagtgaccgtctgctttgcagcttctggcagaggattcatttcttctatctgcacagcatctggcatgtgctcatcagcatcaccttcccttatggcatggtcaccatggccttggtggatgccaactatgagatgccaggtgaaaccctcaaagtccgctactggcctcgggacagttggcccgtggggctgccctacgtggaaatccggggtgatgacaaggactgctga

SEQ ID NO: 14 - DNA, 828 bp, Artificial Sequence
  synthetic nucleotide
atgggcgccccgcactggtgggaccagctgcaggctggtagctcggaggtggactggtgcgaggacaactacaccatcgtgcctgctatcgccgagttctacaacacgatcagcaatgtcttatttttcattttaccgcccatctgcatgtgcttgtttcgtcagtatgcaacatgcttcaacagtggcatctacttaatctggactcttttggttgtagtgggaattggatccgtctacttccatgcaacccttagtttcttgggtcagatgcttgatgaacttgcagtcctttgggttctgatgtgtgctttggccatgtggttccccagaaggtatctaccaaagatctttcggaatgaccggggtaggttcaaggtggtggtcagtgtcctgtctgcggttacgacgtgcctggcatttgtcaagcctgccatcaacaacatctctctgatgaccctgggagttccttgcactgcactgctcatcgcagagctaaagaggtgtgacaacatgcgtgtgtttaagctgggcctcttctcgggcctctggtggaccctggccctgttctgctggatcagtgaccgagctttctgcgagctgctgtcatccttcaacttcccctacctgcactgcatgtggcacatcctcatctgccttgctgcctacctgggctgtgtatgctttgcctactttgatgctgcctcagagattcctgagcaaggccctgtcatcaagttctggcccaatgagaaatgggccttcattggtgtcccctatgtgtccctcctgtgtgccaacaagaaatcatcagtcaagatcacgtga

SEQ ID NO: 15 - DNA, 804 bp, Artificial Sequence
  synthetic nucleotide
atggctccggccgcggaccgagagggctactggggccccacgacctccacgctggactggtgcgaggagaactactccgtgacctggtacatcgccgagttctggaatacagtgagtaacctgatcatgattatacctccaatgttcggtgcagttcagagtgttagagacggtctggaaaagcggtacattgcttcttatttagcactcacagtggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 16 - DNA, 693 bp, Artificial Sequence
  synthetic nucleotide
atggctccggccgcggaccgagagggctactggggccccacgacctccacgctggactggtgcgaggagaactactccgtgacctggtacatcgccgagttcttggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 17 - DNA, 519 bp, Artificial Sequence
  synthetic nucleotide
atgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 18 - DNA, 1965 bp, Artificial Sequence
  synthetic nucleotide
atgaatggacaccttgaagcagaggagcagcaggaccagaggccagaccaggagctgaccgggagctggggccacgggcctaggagcaccctggtcagggctaaggccatggccccgcccccaccgccactggctgccagcaccccgctcctccatggcgagtttggctcctacccagcccgaggcccacgctttgccctcacccttacatcgcaggccctgcacatacagcggctgcgccccaaacctgaagccaggccccggggtggcctggtcccgttggccgaggtctcaggctgctgcaccctgcgaagccgcagcccctcagactcagcggcctacttctgcatctacacctaccctcggggccggcgcggggcccggcgcagagccactcgcaccttccgggcagatggggccgccacctacgaagagaaccgtgccgaggcccagcgctgggccactgccctcacctgtctgctccgaggactgccactgcccggggatggggagatcacccctgacctgctacctcggccgccccggttgcttctattggtcaatccctttgggggtcggggcctggcctggcagtggtgtaagaaccacgtgcttcccatgatctctgaagctgggctgtccttcaacctcatccagacagaacgacagaaccacgcccgggagctggtccaggggctgagcctgagtgagtgggatggcatcgtcacggtctcgggagacgggctgctccatgaggtgctgaacgggctcctagatcgccctgactgggaggaagctgtgaagatgcctgtgggcatcctcccctgcggctcgggcaacgcgctggccggagcagtgaaccagcacgggggatttgagccagccctgggcctcgacctgttgctcaactgctcactgttgctgtgccggggtggtggccacccactggacctgctctccgtgacgctggcctcgggctcccgctgtttctccttcctgtctgtggcctggggcttcgtgtcagatgtggatatccagagcgagcgcttcagggccttgggcagtgcccgcttcacactgggcacggtgctgggcctcgccacactgcacacctaccgcggacgcctctcctacctccccgccactgtggaacctgcctcgcccacccctgcccatagcctgcctcgtgccaagtcggagctgaccctaaccccagacccagccccgcccatggcccactcacccctgcatcgttctgtgtctgacctgcctcttcccctgccccagcctgccctggcctctcctggctcgccagaacccctgcccatcctgtccctcaacggtgggggcccagagctggctggggactggggtggggctggggatgctccgctgtccccggacccactgctgtcttcacctcctggctctcccaaggcagctctacactcacccgtctccgaaggggcccccgtaattcccccatcctctgggctcccacttcccacccctgatgcccgggtaggggcctccacctgcggcccgcccgaccacctgctgcctccgctgggcaccccgctgcccccagactgggtgacgctggagggggactttgtgctcatgttggccatctcgcccagccacctaggcgctgacctggtggcagctccgcatgcgcgcttcgacgacggcctggtgcacctgtgctgggtgcgtagcggcatctcgcgggctgcgctgctgcgccttttcttggccatggagcgtggtagccacttcagcctgggctgtccgcagctgggctacgccgcggcccgtgccttccgcctagagccgctcacaccacgcggcgtgctcacagtggacggggagcaggtggagtatgggccgctacaggcacagatgcaccctggcatcggtacactgctcactgggcctcctggctgcccggggcgggagccctga

SEQ ID NO: 19 - DNA, 22 bp, Artificial Sequence
  synthetic nucleotide
acaggattcaaaccaggactgt

SEQ ID NO: 20 - DNA, 20 bp, Artificial Sequence
  synthetic nucleotide
tgggcatctttccttccgaa

SEQ ID NO: 21 - DNA, 22 bp, Artificial Sequence
  synthetic nucleotide
tgacaggattcaaaccaggact

SEQ ID NO: 22 - DNA, 20 bp, Artificial Sequence
  synthetic nucleotide
ctgggcatctttccttccga

SEQ ID NO: 23 - DNA, 22 bp, Artificial Sequence
  synthetic nucleotide
atactcaccgaacggaagaacc

SEQ ID NO: 24 - DNA, 22 bp, Artificial Sequence
  synthetic nucleotide
ccattagcccattcaccacctc

SEQ ID NO: 25 - DNA, 21 bp, Artificial Sequence
  synthetic nucleotide
actgatactcaccgaacggaa

SEQ ID NO: 26 - DNA, 21 bp, Artificial Sequence
  synthetic nucleotide
cattagcccattcaccacctc

SEQ ID NO: 27 - DNA, 20 bp, Artificial Sequence
  synthetic nucleotide
cacagccaacagtctccaaa

SEQ ID NO: 28 - DNA, 19 bp, Artificial Sequence
  synthetic nucleotide
tctgagtataagccgccca

SEQ ID NO: 29 - DNA, 19 bp, Artificial Sequence
  synthetic nucleotide
atagaccgagcacagccaa

SEQ ID NO: 30 - DNA, 21 bp, Artificial Sequence
  synthetic nucleotide
gaaccttctcaggattgaggt

SEQ ID NO: 31 - DNA, 21 bp, Artificial Sequence
  synthethic nucleotide
taacgaacgagactctggcat

SEQ ID NO: 32 - DNA, 22 bp, Artificial Sequence
  synthetic nucleotide
cggacatctaagggcatcacag